(19) United States
(12) Patent Application Publication
(10) Pub. No.: US 2007/0265646 A1
(43) Pub. Date: Nov. 15, 2007

(54) Dynamically adjustable gastric implants

(75) Inventors: Jay R. McCoy, Temecula, CA (US); Nicholas J. Lembo, Atlanta, GA (US); George F. Kick, Casa Grande, AZ (US); Jay A. Lenker, Laguna Beach, CA (US)

Correspondence Address: MCDERMOTT WILL & EMERY LLP, 18191 VON KARMAN AVE., SUITE 500, IRVINE, CA, 92612-7108, US

(73) Assignee: Ellipse Technologies, Inc., Irvine, CA (US)

(21) Appl. No.: 11/796,502

(22) Filed: Apr. 27, 2007

Related U.S. Application Data
(63) Continuation-in-part of application No. 11/654,068, filed on Jan. 16, 2007.
(60) Provisional application No. 60/796,114, filed on Apr. 27, 2006.
(60) Provisional application No. 60/759,672, filed on Jan. 17, 2006.

(51) Int. Cl. A61B 17/22

(52) U.S. Cl. 606/157

(57) ABSTRACT

Gastric restriction device implants and their use in controlling body weight are described. In some embodiments, activation of a shape memory material drives an actuator coupled to an implant, resulting in a conformational change in the implant. In some embodiments latch and ratchet mechanisms operate incrementally to increase or decrease a size of a stomal opening produced by the gastric restriction device. Methods are described by which adjusting the size of the stomal opening is used to restrict the rate at which food passes through the stomach.

### RELATED APPLICATIONS

This application claims the benefit under 35 U.S.C. § 119(e) of U.S. Provisional Patent Application 60/796,114, entitled “DYNAMICALLY ADJUSTABLE GASTRIC IMPLANTS,” filed Apr. 27, 2006; this application is a continuation-in-part of U.S. patent application Ser. No. 11/654,068, entitled “TWO-WAY ADJUSTABLE IMPLANT,” filed Jan. 16, 2007, and which claims the benefit of U.S. Provisional Patent Application 60/759,672, entitled “TWO-WAY ADJUSTABLE IMPLANT,” filed Jan. 17, 2006; the entirety of all of which are hereby incorporated by reference.

### FIELD OF THE INVENTION

The present invention relates to devices and methods for dynamically restricting the capacity of the stomach using an implant or implants within or around the outside of the stomach and externally or internally activating the implant(s) to induce a change in shape and/or size of the implant(s).

### BACKGROUND OF THE INVENTION

According to the American Society of Bariatric Surgery (ASBS), between 11 and 15 million people in the United States suffer from morbid obesity. Even mild degrees of obesity have adverse health effects and are associated with diminished longevity. For this reason aggressive dietary intervention is recommended. Patients with body mass indices exceeding 40 have medically significant obesity in which the risk of serious health consequences is substantial. For these patients, sustained weight loss rarely occurs with dietary intervention. With the obvious failure of non-operative means of producing permanent weight reduction in patients with morbid obesity, the most effective available treatment is surgery. Surgical treatment is associated with sustained weight loss for the seriously obese patients who uniformly fail non-surgical treatment.

Bariatrics is the branch of medicine concerned with the management of obesity and allied diseases. There are two main categories of bariatric surgery techniques available today. Restrictive techniques reduce the amount of food that can be consumed by restricting the size and/or capacity of the stomach. Malabsorptive techniques alter and/or shorten the digestive tract to decrease the absorption of calories and nutrients. Some surgeries are just restrictive, while others are both restrictive and malabsorptive. A National Institute of Health Consensus Panel reviewed the indications and types of operations and concluded that the banded gastroplasty and gastric bypass were acceptable operations for treating seriously obese patients.

In a Vertical Banded Gastroplasty (“VBG”), or “stomach stapling” procedure, the surgeon staples the upper stomach to create a small, thumb-sized stomach pouch, reducing the quantity of food that the stomach can hold to about 1-2 ounces. The outlet of this pouch is then restricted by a band that significantly slows the emptying of the pouch to the lower part of the stomach. Aside from the creation of a small stomach pouch, there is no other significant change made to the gastrointestinal tract. So while the amount of food the stomach can contain is reduced, the stomach continues to digest nutrients and calories in a normal way. This procedure is purely restrictive; there is no malabsorptive effect. Following this operation, many patients have reported feeling full but not satisfied after eating a small amount of food. As a result, some patients have attempted to get around this effect by eating more or by eating gradually all day long. These practices can result in vomiting, tearing of the staple line, or simply reduced weight loss. Major risks associated with VBG include: unsatisfactory weight loss or weight regain, vomiting, band erosion, band slippage, breakdown of staple line, anastomotic leak, and intestinal obstruction. However, VBG does have the advantage that the body anatomy is left intact and that is completely reversible.

One relatively new and less invasive form of bariatric surgery is Adjustable Gastric Banding. Through this procedure the surgeon places a band around an upper part of the stomach to divide the stomach into two parts, including a small pouch in the upper part of the stomach. The small upper stomach pouch can only hold a small amount of food. The remainder of the stomach lies below the band. The two parts are connected by means of a small opening called a stoma. The stoma is created by placing an adjustable band around the stomach with out stapling to control the size of the stoma. Risks associated with gastric banding are significantly less than other forms of bariatric surgery. As this surgery does not involve opening of the gastric cavity—there is no cutting, stapling or bypassing. The most significant problem associated with the gastric banding has been alteration in the size of the stomach pouch which is isolated above the band. This pouch may enlarge in some cases, either due to slippage of the band, or stretching of the wall of the pouch. In addition, there is the potential for band erosion into the stomach.

The LAP-BAND® Adjustable Gastric Banding System (Inamed) is one current product used in the Adjustable Gastric Banding procedure. The LAP-BAND® system, illustrated in FIG. 1, comprises a silicone band 50, which is essentially an annular-shaped balloon. The surgeon places the silicone band around the upper part of the stomach 52, as described above. The LAP-BAND® system further comprises a port 54 that is placed under the skin, and tubing 56 that provides fluid communication between the port and the band. A physician can inflate the band by injecting a fluid (such as saline) into the band through the port. As the band inflates, the size of the stoma shrinks, thus further limiting the rate at which food can pass from the upper stomach pouch 58 to the lower part of the stomach. The physician can also deflate the band, and thereby increase the size of the stoma, by withdrawing the fluid from the band through the port. The physician inflates and deflates the band by piercing the port, through the skin, with a fine-gauge needle. Disadvantages of this device include the very limited range of adjustment possible with the saline filled balloons, alternate sizes of bands have to be used to cover different sizes of stomachs. Another disadvantage is the invasive manner of adjusting the size of the gastric band by injecting or removing saline from an implanted port below the skin. Infection, erosion of the gastric wall, and slippage of the stomach through the band are additional complications that can arise.

Other examples of dynamically adjustable gastric rings include U.S. patent application Ser. No. 11/351,788, filed on Feb. 10, 2006, entitled “Dynamically Adjustable Gastric Implants and Methods of treating Obesity Using Dynamically Adjustable Gastric Implants,” and incorporated herein in its entirety by reference, which discloses a gastric band comprised at least in part of a shape memory material and configured to transform under the influence of an activation energy from a pre-activation configuration to a post activation configuration.

### SUMMARY OF THE INVENTION

Notwithstanding the foregoing, it would be advantageous to provide a reversible gastric band for creating a stoma opening in the upper part of the stomach in conjunction with a bariatric procedure such that the band may be incrementally and reversibly adjusted to control the size of the stoma opening.

Accordingly, there is provided in some embodiments, an adjustable gastric implant for constraining at least a portion of a stomach, comprising: an elongate member having first and second ends, the elongate member configured to engage the stomach; at least one actuator coupled to the first and second ends of the elongate member, and wherein the at least one actuator comprises a shape memory material; wherein activation of at least a portion of the shape memory material results in a conformational change in the at least one actuator; and wherein the conformational change in the at least one actuator moves the elongate member from a first conformation to a second conformation, such that the first and second ends move with respect to each other, resulting in a change in a lumenal dimension of the stomach.

In some embodiments, placement of the elongate member engages the stomach between an upper region and lower region connected by a stomal lumen.

In some embodiments, moving the elongate member from a first conformation to a second conformation reduces a size of the stomal lumen.

In some embodiments, moving the elongate member from a first conformation to a second conformation increases a size of the stomal lumen.

In some embodiments, the implant is configured to be placed within the stomach.

In some embodiments, the implant is configured to be placed around an outer surface of the stomach.

In some embodiments, the activation comprises application of an energy to the shape memory material.

In some embodiments, the energy is at least one of ultrasound energy, radio frequency energy, X-ray energy, microwave energy, light, electric field energy, magnetic field energy, inductive heating, or conductive heating.

In some embodiments, there is provided an adjustable gastric implant to implant around at least a portion of the stomach, comprising: a elongate member having first and second ends; a latch mounted on the first end of the elongate member and configured to engage the second end of the elongate member; an actuator coupled to the latch, the actuator configured to advance the second end of the elongate member within the latch; wherein the actuator comprises a shape memory component, the shape memory component configured to result in a conformational change in the actuator.

In some embodiments, under the influence of an activation energy, the shape memory component drives the actuator from a first conformation to a second conformation, the conformational change effective to advance the second end of the elongate member within the latch.

In some embodiments, the activation energy comprises at least one of ultrasound energy, radio frequency energy, X-ray energy, microwave energy, light, electric field energy, magnetic field energy, inductive heating, or conductive heating.

In some embodiments, the implant further comprises an induction coil assembly having a transmission element connected to the latch, the assembly coil configured to deliver the activation energy to the at least one shape memory component via the transmission element.

In some embodiments, the implant further comprises a disengagement member, comprising: a second shape memory component, configured such that in response to a second activation energy, the second shape memory component changes conformation, resulting in the actuator disengaging from the latch; and a bias member, effective to withdraw at least a portion of the elongate member from the latch when the actuator is disengaged from the latch.

In some embodiments, the implant further comprises: a third actuator operably coupled to the latch; wherein the third actuator comprises a shape memory element; wherein in response to an activation energy, the third actuator changes from a first conformation to a second conformation; and wherein the change in conformation of the third actuator results in at least a portion of the second end of the elongate member being withdrawn from the latch.

In some embodiments, the implant further comprises a stop, configured to prevent complete withdrawal of the elongate member from the latch.

In some embodiments, the implant further comprises a position sensor, operative to sense a position of the second end of the elongate member relative to a position of the latch.

In some embodiments, the position sensor comprises a magnetic sensor mounted on the latch, and a magnetic member mounted on the elongate member, and wherein the magnetic sensor senses the relative position of the magnetic member.

In some embodiments, the implant further comprises at least one silicone pad disposed along at least a portion of the length of the elongate member.

In some embodiments, the implant further comprises an attachment mechanism effective to secure the implant at a desired location in the body.

In some embodiments, the attachment mechanism comprises at least one of a suture hole, a suture ring, a hook, a barb, and an anchor.

In some embodiments, the desired location in the body is around at least a portion of an outer surface of the stomach.

In some embodiments, the desired location in the body is within the stomach.

In some embodiments, the shape memory component comprises at least one of a metal, a metal alloy, a nickel titanium alloy, and a shape memory polymer.

In some embodiments, the shape memory component comprises at least one of Fe—C, Fe—Pd, Fe—Mn—Si, Co—Mn, Fe—Co—Ni—Ti, Ni—Mn—Ga, Ni₂MnGa, and Co—Ni—Al.

In some embodiments, the elongate member comprises a biocompatible plastic.

In some embodiments, during a vertical banded gastroplasty procedure, the implant is configured to constrain at least a portion of the greater curvature of the stomach, by drawing at least a portion of the second end of the elongate member through the latch.

In some embodiments, a surface of the elongate member further comprises a plurality of detents, configured to reversibly engage the latch; wherein the actuator is configured to advance incrementally the elongate member into the latch by a distance approximately equal to a distance between adjacent detents each time the shape memory component is subjected to an effective amount of the activation energy.

In some embodiments, the implant further comprises a second actuator operably coupled to the latch, the second actuator comprising a second shape memory component; wherein activation of the second shape memory component by a second activation energy results in the second shape memory component undergoing a conformational change that is effective to drive the second actuator; wherein driving the second actuator withdraws incrementally the elongate member from the latch; and wherein each time the second shape memory component is subjected to an effective amount of the second activation energy, the elongate member is withdrawn by a distance approximately equal to a distance between adjacent detents.

In some embodiments there is provided an adjustable gastric implant configured to constrain at least a portion of the stomach, comprising: an elongate member having first end and second ends; latching means that couples the first and second ends of the elongate member, such that the elongate member is maintained in a shape of a substantially closed loop; and ratcheting means comprising a shape memory component, the ratcheting means configured to engage an end of the elongate member; and wherein, in response to an activation energy, the shape memory component undergoes a conformational change effective to result in the ratcheting means advancing the elongate member within the latching means.

In some embodiments, the elongate member further comprises stop means configured to prevent the elongate member from being fully withdrawn from the latching means.

In some embodiments, the implant further comprises a bias means effective to withdraw at least a portion of the elongate member from the latching means.

In some embodiments, the implant further comprises: a second ratcheting means comprising a second shape memory component; the second ratcheting means configured to engage an opposite end of the elongate member; and wherein, in response to an activation energy, the second shape memory component undergoes a conformational change effective to result in the second ratcheting means advancing the elongate member within a second latching means.

In some embodiments, the ratcheting means further comprises a third shape memory component; wherein in response to a third activation energy, the third shape memory component is configured to result in the ratcheting means releasing the engaged end of elongate member.

In some embodiments, the ratcheting means further comprises a third shape memory component; wherein in response to a third activation energy, the third shape memory component is configured to result in the first ratcheting means releasing the engaged end of elongate member.

In some embodiments, the second ratcheting means further comprises a fourth shape memory component, and wherein the third and fourth shape memory components are configured such that in response to an activation energy, at least one of the first and second ends of the elongate member is released from the latching means.

In some embodiments there is provided, a method of regulating food intake in a patient, comprising the steps of: providing an adjustable gastric implant comprising an elongate member coupled to an actuator having a shape memory component; placing the implant to engage at least a portion of the stomach between an upper region and a lower region connected by a stomal opening; applying an activation energy to the shape memory component; wherein application of the activation energy transforms the shape memory component from a first conformation to a second conformation, said transformation effective to drive the actuator; and wherein driving the actuator results in a conformational change in the implant such that the diameter of the stomal opening is decreased; and wherein decreasing the diameter of the stomal opening reduces the rate at which food passes through the stomach.

In some embodiments, the method further comprises reconfiguring the shape memory component from the second conformation back to the first conformation.

In some embodiments, the method further comprises alternating the conformation of the shape memory component between the first and second configurations to decrease incrementally a diameter of the stomal opening.

In some embodiments of the method, the actuator engages the ends of the elongate member to form a substantially closed loop.

In some embodiments of the method, the implant further comprises a bias member, and the method further comprises disengaging at least one end of the elongate member from the actuator, such that the bias member is effective to increase the perimeter of the closed loop formed by the elongate member to a maximal perimeter.

In some embodiments of the method, the disengaging further comprises activating a second shape memory component on the actuator, thereby disengaging the actuator.

In some embodiments of the method, the implant further comprises a second actuator having a third shape memory component, the second actuator coupled to the elongate member, the method further comprising: applying an activation energy to the third shape memory component; wherein application of the activation energy results in the third shape memory component being transformed from a first conformation to a second conformation; and wherein transformation of the third shape memory component drives the second actuator to expand a perimeter of the loop resulting in an increase in the diameter of the stomal opening, thereby increasing a rate at which food can pass through the stomach.

In some embodiments of the method, the shape memory component of the implant comprises at least one of a metal, a metal alloy, a nickel titanium alloy, and a shape memory polymer.

In some embodiments of the method, a shape memory component of the implant comprises at least one of Fe—C, Fe—Pd, Fe—Mn—Si, Co—Mn, Fe—Co—Ni—Ti, Ni—Mn—Ga, Ni₂MnGa, and Co—Ni—Al.

In some embodiments of the method, the activation energy comprises at least one of magnetic resonance imaging energy, high-intensity focused ultrasound energy, radio frequency energy, x-ray energy, microwave energy, light energy, electric field energy, magnetic field energy, inductive heating, and conductive heating.

In some embodiments there is provided a method of adjusting a gastric implant in a patient, comprising; placing an adjustable gastric implant around at least a portion of the stomach of the patient; adjusting the implant to produce a constriction of the stomach; using an imaging technique to determine a first size of the constriction; and adjusting the gastric restriction device to vary the constriction to a second size and limit the rate at which food passes through the constriction.

In some embodiments of the method, the imaging technique comprises at least one of MRI, X-ray fluoroscopy, and ultrasound imaging.

In some embodiments of the method, the imaging technique comprises an ultrasound technique that uses speed of sound shift.

### BRIEF DESCRIPTION OF THE DRAWINGS

The preferred embodiments of the present gastric implants and methods, illustrating their features, will now be discussed in detail. These embodiments depict the novel and non-obvious gastric implants shown in the accompanying drawings, which are for illustrative purposes only. These drawings include the following figures, in which like numerals indicate like parts.

FIG. 1 is a front elevational view of a stomach that has undergone a Gastric Banding procedure using the prior art LAP-BAND® Adjustable Gastric Banding System.

FIG. 2 is a front elevational view of a stomach that has undergone a Gastric Banding procedure using one embodiment of the present dynamically adjustable gastric implants.

FIG. 3 is a front elevational view of the stomach of FIG. 2 after the implant has been adjusted.

FIG. 4 is a front elevational view of a stomach that has undergone a Gastric Banding procedure using another embodiment of the present dynamically adjustable gastric implants.

FIG. 5 is a front perspective view of one embodiment of the present dynamically adjustable gastric implants.

FIG. 6 is a front perspective view of the implant of FIG. 5 after the implant has been adjusted.

FIG. 7 is a front perspective view of the implant of FIG. 5 after the implant has been further adjusted from the configuration of FIG. 6.

FIG. 8 is a top plan view of another embodiment of the present dynamically adjustable gastric implants, illustrating the implant in a pre-adjusted configuration.

FIG. 9 is a top plan view of the implant of FIG. 8, illustrating the implant in a post-adjusted configuration.

FIG. 10 is a top plan view of another embodiment of the present dynamically adjustable gastric implants.

FIG. 11 is a top plan view of another embodiment of the present dynamically adjustable gastric implants.

FIG. 12 is a top plan view of another embodiment of the present dynamically adjustable gastric implants.

FIG. 13 is a top plan view of another embodiment of the present dynamically adjustable gastric implants.

FIG. 14 is a top plan view of another embodiment of the present dynamically adjustable gastric implants.

FIG. 15 is a detail view of the portion of the implant of FIG. 14 indicated by the line 15-15.

FIG. 16 is a top plan view of another embodiment of the present dynamically adjustable gastric implants, illustrating the implant in a pre-adjusted configuration.

FIG. 17 is a top plan view of the implant of FIG. 16, illustrating the implant in a post-adjusted configuration.

FIG. 18 is a top plan view of the implant of FIGS. 16 and 17, illustrating the pre-adjusted and post-adjusted configurations superimposed upon one another.

FIG. 19 is a top plan view of another embodiment of the present dynamically adjustable gastric implants, illustrating the implant in a pre-adjusted configuration.

FIG. 20 is a top plan view of the implant of FIG. 19, illustrating the implant in a post-adjusted configuration.

FIG. 21 is a front elevational view of another embodiment of the present dynamically adjustable gastric implants and a stomach, illustrating a configuration of the implant and stomach after activation of the implant.

FIG. 22 is a front elevational view of another embodiment of the present dynamically adjustable gastric implants and a stomach, illustrating a configuration of the implant and stomach after activation of the implant.

FIG. 23 is a front elevational view of another embodiment of the present dynamically adjustable gastric implants and a stomach, illustrating a configuration of the implant and stomach after activation of the implant.

FIG. 24 is a top plan view of another embodiment of the present dynamically adjustable gastric implants, illustrating the implant in a pre-adjusted configuration.

FIG. 25 is a top plan view of the implant of FIG. 24, illustrating the implant in a post-adjusted configuration.

FIG. 26 is a front perspective view of another embodiment of the present dynamically adjustable gastric implants.

FIG. 27 is a front elevational view of another embodiment of the present dynamically adjustable gastric implants.

FIG. 28 is a front elevational view of another embodiment of the present dynamically adjustable gastric implants, illustrating several different sizes of the embodiment.

FIG. 29 is a front perspective view of another embodiment of the present dynamically adjustable gastric implants.

FIG. 30 is a front elevational view of a stomach and esophagus, illustrating schematically one possible configuration for implantation of any of the implants of FIGS. 26-29.

FIG. 31 is a detail view of a portion of another embodiment of the present dynamically adjustable gastric implants.

FIG. 32 is a detail view of the portion of FIG. 31 after the implant has been adjusted.

FIG. 33 is a front elevational view of a patient and another embodiment of the present dynamically adjustable gastric implants, illustrating one method of adjusting the implant using direct application of electrical impulses.

FIG. 34 is a front elevational view of one step in a method of implanting any of the present implants using a balloon catheter.

FIG. 35 is a front elevational view of a stomach that has undergone a Vertical Gastric Banding procedure using one embodiment of the present dynamically adjustable gastric implants.

FIG. 36 is a top view of one embodiment of the present dynamically adjustable gastric implants in a pre-implantation configuration.

FIG. 37 is the side view of one embodiment of the present dynamically adjustable gastric implants in a post implantation configuration.

FIG. 38 is a side view of one embodiment of an adjustable gastric band.

FIG. 39 is a bottom view of one embodiment of an adjustable gastric band showing the leaf spring.

FIG. 40 is a side view of an embodiment of the latch head mechanism for the gastric band of FIG. 38.

FIG. 41 is a top view of an embodiment of the latch head mechanism for the gastric band of FIG. 38.

FIG. 42 is an end view of an embodiment of the latch head mechanism for the gastric band of FIG. 38.

FIG. 43 is a side view of one embodiment of an adjustable gastric band showing the induction coil.

FIG. 44 is a side view of the induction coil of the gastric band of FIG. 43.

FIG. 45 is a side view of one embodiment of an adjustable gastric band.

FIG. 46 is a top view of an embodiment of the latch head mechanism for the gastric band of FIG. 45.

FIG. 47 is a side view of an embodiment of the latch head mechanism for the gastric band of FIG. 45.

FIG. 48 is an end view of an embodiment of the latch head mechanism for the gastric band of FIG. 45:

FIG. 49 is a top view of the band for the gastric band of FIG. 45 showing a position sensor.

FIG. 50 is a side view of one embodiment of an adjustable gastric band showing the induction coil.

FIG. 51 is a side view of the induction coil of the gastric band of FIG. 50.

### DETAILED DESCRIPTION OF THE INVENTION

The present invention includes gastric implants and methods for dynamically restricting the capacity of a patient's stomach to treat obesity. As used herein, the term “gastric implant” describes an implant or implants that are configured for implantation within or around the outside of the stomach. Such implants are further configured to be dynamically adjusted, for example, by externally or internally activating the implant(s) to induce a change in shape and/or size of the implant(s).

In certain embodiments, the band may be placed within the stomach. The band may be placed fully or partially within the stomach. The device may further comprise suture rings or holes where device is attached to the stomach tissue. Once implant is secured to the stomach, post implant activation will cause the shrinkage or expansion of implant and the applied force will push the stomach to expand or shrink accordingly. In yet another embodiment, suturing or securing of implant to the tissue can be done by variety of techniques such as: automatic stapling, manual stapling, tissue glue, heat activated glue, UV curing glue, room temperature or moisture activated glue. In yet another embodiment, suturing or securing of implant to the tissue can be done by variety of mechanical fastening techniques such as hooks, anchors, or barbs. In yet another embodiment, securing and suturing of implant to the tissue can be done by other energy sources such as: RF heating, laser, Microwave, Ultrasound, etc. In yet another embodiment, securing and suturing of implant to the tissue can be done all around the implant perimeter or at one or more points or segments.

The size and/or configuration of the implant may be adjusted post-implantation through one of many techniques, including minimally invasive techniques and completely non-invasive techniques. For example, minimally invasive techniques include endoscopic, laparoscopic, percutaneous, etc. Completely non-invasive techniques include magnetic resonance imaging (MRI), application of high-intensity focused ultrasound (HIFU), inductive heating, a combination of these methods, etc. The implant may be adjusted at a time shortly after implantation in order to constrict and/or expand the outlet from the stomach pouch to the rest of the stomach. The implant may also be adjusted at a later time in order to further constrict and/or expand the outlet. As used herein, “post-implantation” refers to a time after implanting the implant and closing the body opening through which the implant was introduced into the patient's body.

In certain embodiments, the implant comprises a shape memory material that is responsive to changes in temperature and/or exposure to a magnetic field. Shape memory is the ability of a material to regain its shape after deformation. Shape memory materials include polymers, metals, metal alloys and ferromagnetic alloys. The implant may be adjusted in vivo by applying an energy source to activate the shape memory material and cause it to change to a memorized shape. The energy source may include, for example, radio frequency (RF) energy, x-ray energy, microwave energy, ultrasonic energy such as focused ultrasound, HIFU energy, light energy, electric field energy, magnetic field energy, combinations of the foregoing, or the like. For example, one embodiment of electromagnetic radiation that is useful is infrared energy having a wavelength in a range between approximately 750 nanometers and approximately 1600 nanometers. This type of infrared radiation may be produced efficiently by a solid state diode laser. In certain embodiments, the shape memory material on the implant may be selectively heated using short pulses of energy having an on and off period between each cycle. The energy pulses provide segmental heating, which allows segmental adjustment of portions of the implant without adjusting the entire implant.

In certain embodiments, the implant may include an energy absorbing material to increase heating efficiency and localize heating in the area of the shape memory material. Thus, damage to the surrounding tissue can be reduced or eliminated. Energy absorbing materials for light or laser activation energy may include nanoshells, nanospheres, and the like, particularly where infrared laser energy is used to energize the material. Such nanoparticles may be made from a dielectric, such as silica, coated with an ultra thin layer of a conductor, such as gold, and be selectively tuned to absorb a particular frequency of electromagnetic radiation. In certain such embodiments, the nanoparticles range in size between about 5 nanometers and about 20 nanometers and can be suspended in a suitable material or solution, such as saline solution. Coatings comprising nanotubes or nanoparticles can also be used to absorb energy from, for example, HIFU, MRI, inductive heating, or the like. In the case of MRI, the coating might include a specific resonance frequency other than the 64 MHz that is typically used in MRI. Thus, the implant can be imaged and controllably adjusted in size and/or shape by using two or more different frequencies of energy simultaneously. A tuneable frequency can be used to better direct activation energy without impacting the image quality.

In other embodiments, thin film deposition or other coating techniques such as sputtering, reactive sputtering, metal ion implantation, physical vapor deposition, and chemical deposition can be used to cover portions or all of the implant. Such coatings can be either solid or microporous. When HIFU energy is used, for example, a microporous structure may trap and direct the HIFU energy toward the shape memory material. The coating improves thermal conduction and heat removal. In certain embodiments, the coating also enhances radio-opacity of the implant. Coating materials can be selected from various groups of biocompatible organic or non-organic, metallic or non-metallic materials such as titanium nitride (TiN), iridium oxide (Irox), carbon, graphite, ceramic, platinum black, titanium carbide (TiC) and other materials used for pacemaker electrodes or implantable pacemaker leads. Other materials discussed herein or known in the art can also be used to absorb energy.

In addition, or in other embodiments, fine conductive wires such as platinum coated copper, titanium, tantalum, stainless steel, gold, or the like, may be wrapped around the shape memory material to allow focused and rapid heating of the shape memory material while reducing undesired heating of surrounding tissues.

In certain embodiments, the energy source is applied surgically either during implantation or at a later time. For example, the shape memory material can be heated during implantation of the implant by touching the implant with a warm object. As another example, the energy source can be surgically applied after the implant has been implanted by inserting a catheter into the patient's body and applying the energy through the catheter. The catheter may be inserted percutaneously, or through a peroral transgastric procedure, for example. Various types of energy, such as ultrasound, microwave energy, RF energy, light energy or thermal energy (e.g., from a heating element using resistance heating), can be transferred to the shape memory material through a catheter positioned on or near the shape memory material. Alternatively, thermal energy can be provided to the shape memory material by injecting a heated fluid through a catheter or circulating the heated fluid in a balloon through the catheter placed in close proximity to the shape memory material. As another example, the shape memory material can be coated with a photodynamic absorbing material that is activated to heat the shape memory material when illuminated by light from a laser diode or directed to the coating through fiber optic elements in a catheter. In certain such embodiments, the photodynamic absorbing material includes one or more drugs that are released when illuminated by the laser light.

In certain embodiments, a removable subcutaneous electrode or coil couples energy from a dedicated activation unit. In certain such embodiments, the removable subcutaneous electrode provides telemetry and power transmission between the system and the implant. The subcutaneous removable electrode allows more efficient coupling of energy to the implant with minimum or reduced power loss. In certain embodiments, the subcutaneous energy is delivered via inductive coupling.

In other embodiments, the energy source is applied in a non-invasive manner from outside the patient's body. In certain such embodiments, the external energy source may be focused to provide directional heating to the shape memory material so as to reduce or minimize damage to the surrounding tissue. For example, in certain embodiments, a handheld or portable device comprising an electrically conductive coil generates an electromagnetic field that non-invasively penetrates the patient's body and induces a current in the implant. The current heats the implant and causes the shape memory material to transform to a memorized shape. In certain such embodiments, the implant may also comprise an electrically conductive coil wrapped around or embedded in the shape memory material. The externally generated electromagnetic field induces a current in the implant's coil, causing it to heat and transfer thermal energy to the shape memory material.

In certain other embodiments, an external HIFU transducer focuses ultrasound energy onto the implant to heat the shape memory material. In certain such embodiments, the external HIFU transducer is a handheld or portable device. The terms “HIFU,” “high intensity focused ultrasound” or “focused ultrasound” as used herein are broad terms and are used at least in their ordinary sense and include, without limitation, acoustic energy within a wide range of intensities and/or frequencies. For example, HIFU includes acoustic energy focused in a region, or focal zone, having an intensity and/or frequency that is considerably less than what is currently used for ablation in medical procedures. Thus, in certain such embodiments, the focused ultrasound is not destructive to the patient's organ tissue. In certain embodiments, HIFU includes acoustic energy within a frequency range of approximately 0.5 MHz and approximately 30 MHz and a power density within a range of approximately 1 W/cm² and approximately 500 W/cm².

In certain embodiments, the implant comprises an ultrasound absorbing material or hydro-gel material that allows focused and rapid heating when exposed to the ultrasound energy and transfers thermal energy to the shape memory material. In certain embodiments, a HIFU probe is used with an adaptive lens to compensate for movement within the body due to, for example, respiration. The adaptive lens has multiple focal point adjustments. In certain embodiments, a HIFU probe with adaptive capabilities comprises a phased array or linear configuration. In certain embodiments, an external HIFU probe comprises a lens configured to be placed between a patient's ribs to improve acoustic window penetration and reduce or minimize issues and challenges regarding passing through bones.

In certain embodiments, HIFU or other activation energy can be synchronized with an imaging device, such as MRI, ultrasound or X-ray, to allow visualization of the implant during HIFU activation. The imaging device may include an algorithm to display the area of interest for energy delivery. In addition, or in other embodiments, ultrasound imaging can be used to non-invasively monitor the temperature of tissue surrounding the implant by using principles of speed of sound shift and changes to tissue thermal expansion.

In certain embodiments, non-invasive energy is applied to the implant post-implantation using a Magnetic Resonance Imaging (MRI) device. In certain such embodiments, the shape memory material is activated by a constant magnetic field generated by the MRI device. In addition, or in other embodiments, the MRI device generates RF pulses that induce current in the implant and heat the shape memory material. The implant can include one or more coils and/or MRI energy absorbing material to increase the efficiency and directionality of the heating. Suitable energy absorbing materials for magnetic activation energy include particulates of ferromagnetic material. Suitable energy absorbing materials for RF energy include ferrite materials as well as other materials configured to absorb RF energy at resonant frequencies thereof.

In certain embodiments, the MRI device is used to determine the size of the implanted implant before, during and/or after the shape memory material is activated. In certain such embodiments, the MRI device generates RF pulses at a first frequency to heat the shape memory material and at a second frequency to image the implant. Thus, the size of the implant can be measured without heating the implant. In certain such embodiments, an MRI energy absorbing material heats sufficiently to activate the shape memory material when exposed to the first frequency and does not substantially heat when exposed to the second frequency. Other imaging techniques known in the art can also be used to determine the size of the implant including, for example, ultrasound imaging, computed tomography (CT) scanning, X-ray imaging, or the like. In certain embodiments, such imaging techniques also provide sufficient energy to activate the shape memory material.

As discussed above, shape memory materials include, for example, polymers, metals, and metal alloys including ferromagnetic alloys. Examples of shape memory polymers that are usable for certain embodiments of the present implant are disclosed by Langer, et al. in U.S. Pat. No. 6,720,402, issued Apr. 13, 2004, U.S. Pat. No. 6,388,043, issued May 14, 2002, and 6,160,084, issued Dec. 12, 2000, each of which are hereby incorporated by reference herein. Shape memory polymers respond to changes in temperature by changing to one or more permanent or memorized shapes. In certain embodiments, the shape memory polymer may be heated to a temperature between approximately 38 degrees Celsius and approximately 60 degrees Celsius. In certain other embodiments, the shape memory polymer may be heated to a temperature in a range between approximately 40 degrees Celsius and approximately 55 degrees Celsius. In certain embodiments, the shape memory polymer has a two-way shape memory effect wherein the shape memory polymer can be heated to change it to a first memorized shape and cooled to change it to a second memorized shape. The shape memory polymer can be cooled, for example, by inserting or circulating a cooled fluid through a catheter.

Shape memory polymers implanted in a patient's body can be heated non-invasively using, for example, external light energy sources such as infrared, near-infrared, ultraviolet, microwave and/or visible light sources. Preferably, the light energy is selected to increase absorption by the shape memory polymer and reduce absorption by the surrounding tissue. Thus, damage to the tissue surrounding the shape memory polymer is reduced when the shape memory polymer is heated to change its shape. In other embodiments, the shape memory polymer comprises gas bubbles or bubble containing liquids such as fluorocarbons and is heated by inducing a cavitation effect in the gas/liquid when exposed to HIFU energy. In other embodiments, the shape memory polymer may be heated using electromagnetic fields and may be coated with a material that absorbs electromagnetic fields.

Certain metal alloys have shape memory qualities and respond to changes in temperature and/or exposure to magnetic fields. Examples of shape memory alloys that respond to changes in temperature include titanium-nickel, copper-zinc-aluminum, copper-aluminum-nickel, iron-manganese-silicon, iron-nickel-aluminum, gold-cadmium, combinations of the foregoing, and the like. In certain embodiments, the shape memory alloy comprises a biocompatible material such as a titanium-nickel alloy.

Shape memory alloys exist in two distinct solid phases called martensite and austenite. The martensite phase is relatively soft and easily deformed, whereas the austenite phase is relatively stronger and less easily deformed. For example, shape memory alloys enter the austenite phase at a relatively high temperature and the martensite phase at a relatively low temperature. Shape memory alloys begin transforming to the martensite phase at a start temperature (Mₛ) and finish transforming to the martensite phase at a finish temperature (M_{f}). Similarly, such shape memory alloys begin transforming to the austenite phase at a start temperature (Aₛ) and finish transforming to the austenite phase at a finish temperature (A_{f}). Both transformations have a hysteresis. Thus, the Mₛ temperature and the A_{f} temperature are not coincident with each other, and the M_{f} temperature and the Aₛ temperature are not coincident with each other.

In certain embodiments, the shape memory alloy is processed to form a memorized shape in the austenite phase in the form of a ring or partial ring. The shape memory alloy is then cooled below the M_{f} temperature to enter the martensite phase and deformed into a larger or smaller ring. In certain such embodiments, the shape memory alloy is sufficiently malleable in the martensite phase to allow a user such as a physician to adjust the circumference of the ring in the martensite phase by hand to achieve a desired fit for a particular stomach. After the ring is attached to the stomach, the circumference of the ring can be adjusted non-invasively by heating the shape memory alloy to an activation temperature (e.g., temperatures ranging from the Aₛ temperature to the A_{f} temperature).

Thereafter, when the shape memory alloy is exposed to a temperature elevation and transformed to the austenite phase, the alloy changes in shape from the deformed shape to the memorized shape. Activation temperatures at which the shape memory alloy causes the shape of the implant to change shape can be selected and built into the implant such that collateral damage is reduced or eliminated in tissue adjacent the implant during the activation process. Examples of A_{f} temperatures for suitable shape memory alloys range between approximately 45 degrees Celsius and approximately 70 degrees Celsius. Furthermore, examples of Mₛ temperatures range between approximately 10 degrees Celsius and approximately 20 degrees Celsius, and examples of M_{f} temperatures range between approximately −1 degrees Celsius and approximately 15 degrees Celsius. The size of the implant can be changed all at once or incrementally in small steps at different times in order to achieve the adjustment necessary to produce the desired clinical result.

Certain shape memory alloys may further include a rhombohedral phase, having a rhombohedral start temperature (Rₛ) and a rhombohedral finish temperature (R_{f}), that exists between the austenite and martensite phases. An example of such a shape memory alloy is a NiTi alloy, which is commercially available from Memry Corporation (Bethel, Conn.). In certain embodiments, an example of an Rₛ temperature range is between approximately 30 degrees Celsius and approximately 50 degrees Celsius, and an example of an R_{f} temperature range is between approximately 20 degrees Celsius and approximately 35 degrees Celsius. One benefit of using a shape memory material having a rhombohedral phase is that in the rhomobohedral phase the shape memory material may experience a partial physical distortion, as compared to the generally rigid structure of the austenite phase and the generally deformable structure of the martensite phase.

Certain shape memory alloys exhibit a ferromagnetic shape memory effect wherein the shape memory alloy transforms from the martensite phase to the austenite phase when exposed to an external magnetic field. The term “ferromagnetic” as used herein is a broad term and is used in its ordinary sense and includes, without limitation, any material that easily magnetizes, such as a material having atoms that orient their electron spins to conform to an external magnetic field. Ferromagnetic materials include permanent magnets, which can be magnetized through a variety of modes, and materials, such as metals, that are attracted to permanent magnets. Ferromagnetic materials also include electromagnetic materials that are capable of being activated by an electromagnetic transmitter, such as one located outside the stomach. Furthermore, ferromagnetic materials may include one or more polymer-bonded magnets, wherein magnetic particles are bound within a polymer matrix, such as a biocompatible polymer. The magnetic materials can comprise isotropic and/or anisotropic materials, such as for example NdFeB (neodymium-iron-boron), SmCo (samarium-cobalt), ferrite and/or AlNiCo (aluminum-nickel-cobalt) particles.

Thus, an implant comprising a ferromagnetic shape memory alloy can be implanted in a first configuration having a first shape and later changed to a second configuration having a second (e.g., memorized) shape without heating the shape memory material above the Aₛ temperature. Advantageously, nearby healthy tissue is not exposed to high temperatures that could damage the tissue. Further, since the ferromagnetic shape memory alloy does not need to be heated, the size of the implant can be adjusted more quickly and more uniformly than by heat activation.

Examples of ferromagnetic shape memory alloys include Fe—C, Fe—Pd, Fe—Mn—Si, Co—Mn, Fe—Co—Ni—Ti, Ni—Mn—Ga, Ni₂MnGa, Co—Ni—Al, and the like. Certain of these shape memory materials may also change shape in response to changes in temperature. Thus, the shape of such materials can be adjusted by exposure to a magnetic field, by changing the temperature of the material, or both.

In certain embodiments, combinations of different shape memory materials are used. For example, implants according to certain embodiments comprise a combination of shape memory polymer and shape memory alloy (e.g., NiTi). In certain such embodiments, an implant comprises a shape memory polymer tube and a shape memory alloy (e.g., NiTi) disposed within the tube. Such embodiments are flexible and allow the size and shape of the implant to be further reduced without impacting fatigue properties. In addition, or in other embodiments, shape memory polymers are used with shape memory alloys to create a bi-directional (e.g., capable of expanding and contracting) implant. Bi-directional implants can be created with a wide variety of shape memory material combinations having different characteristics.

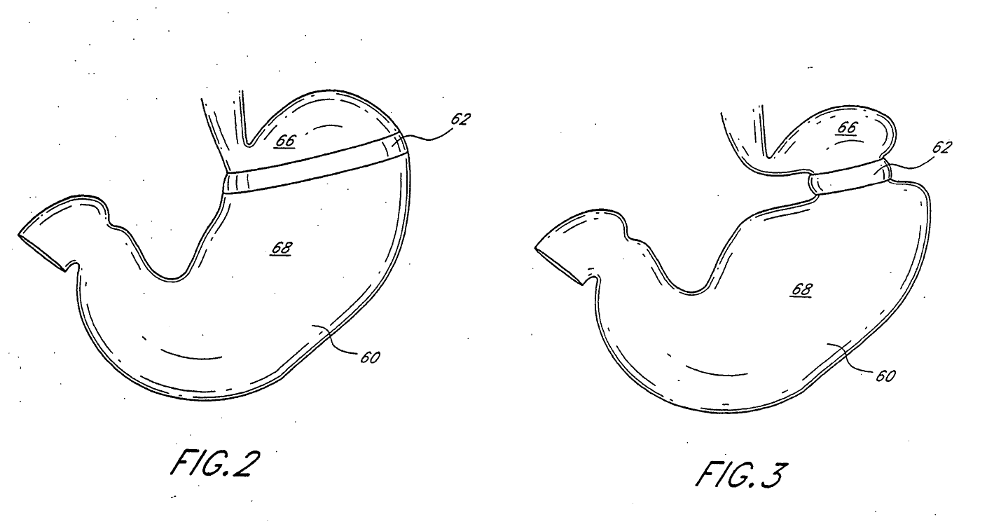

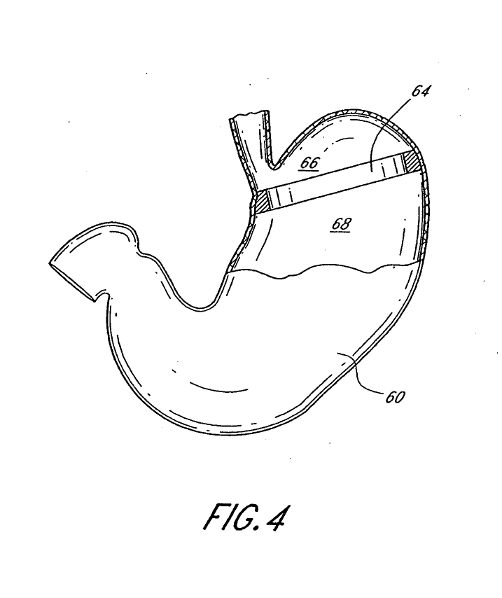

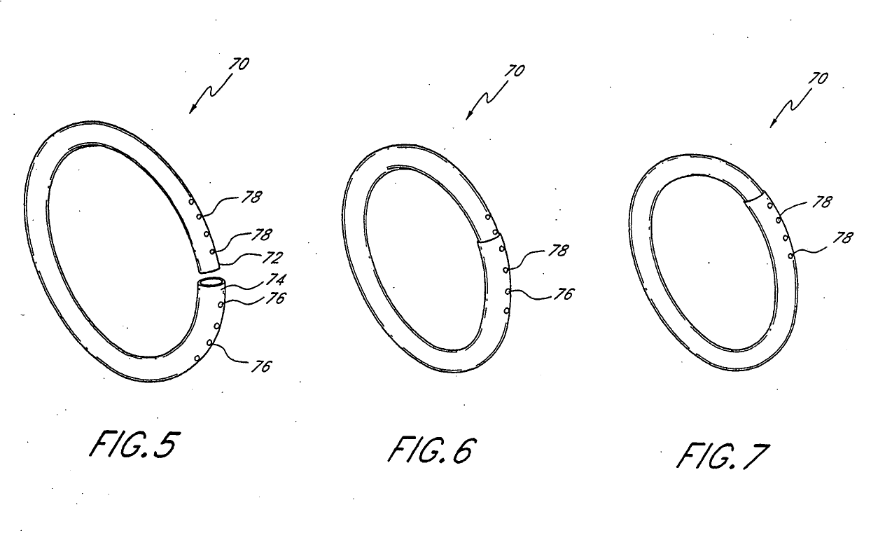

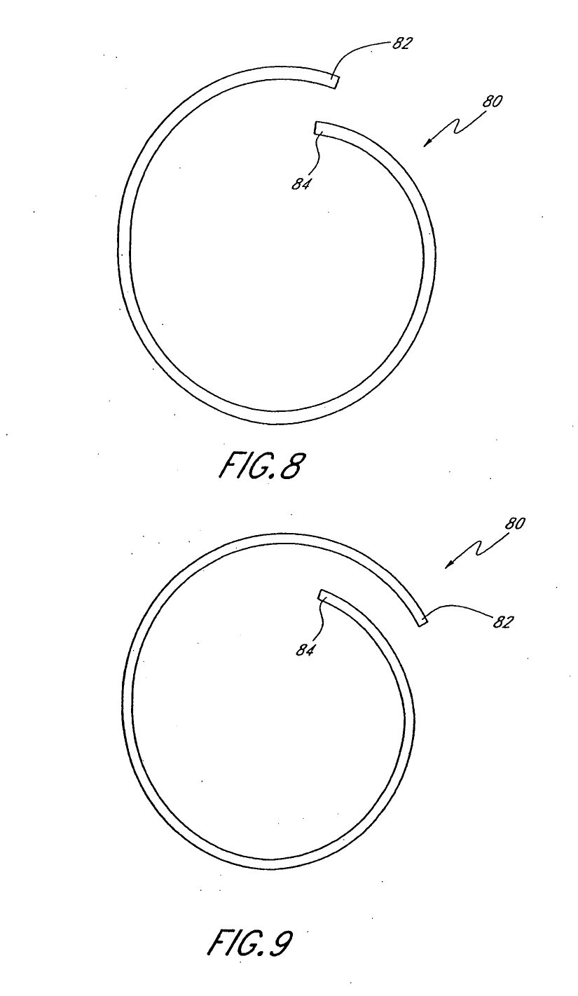

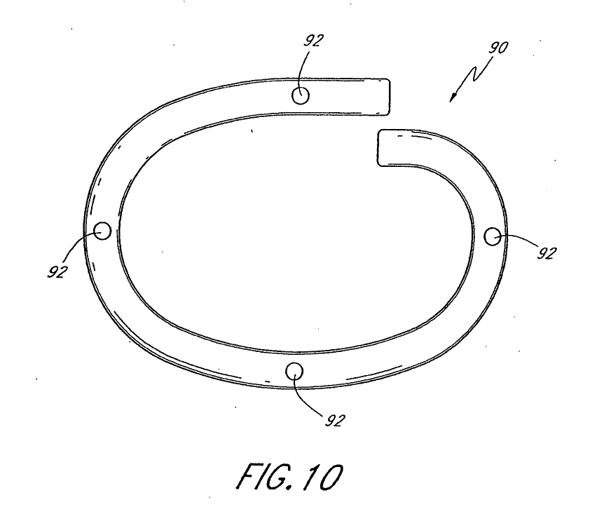

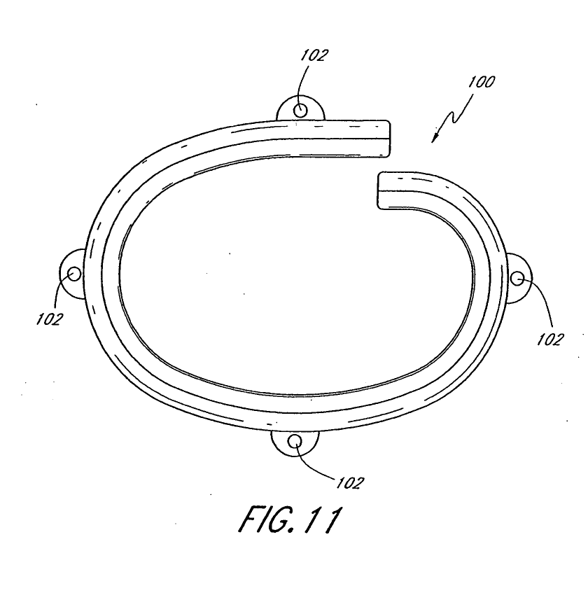

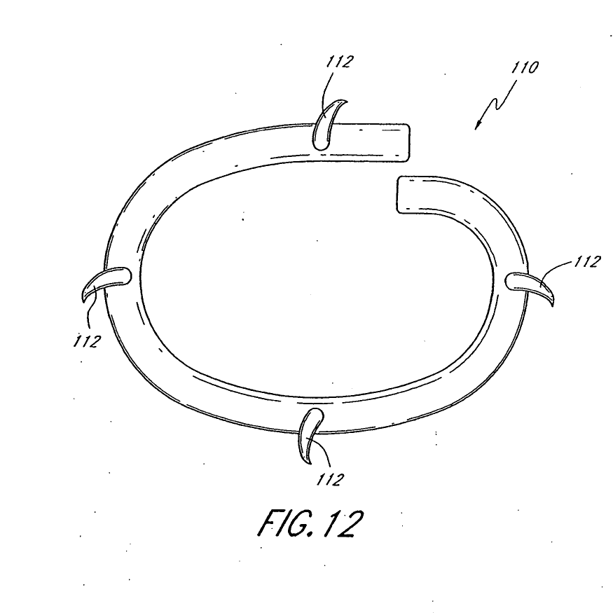

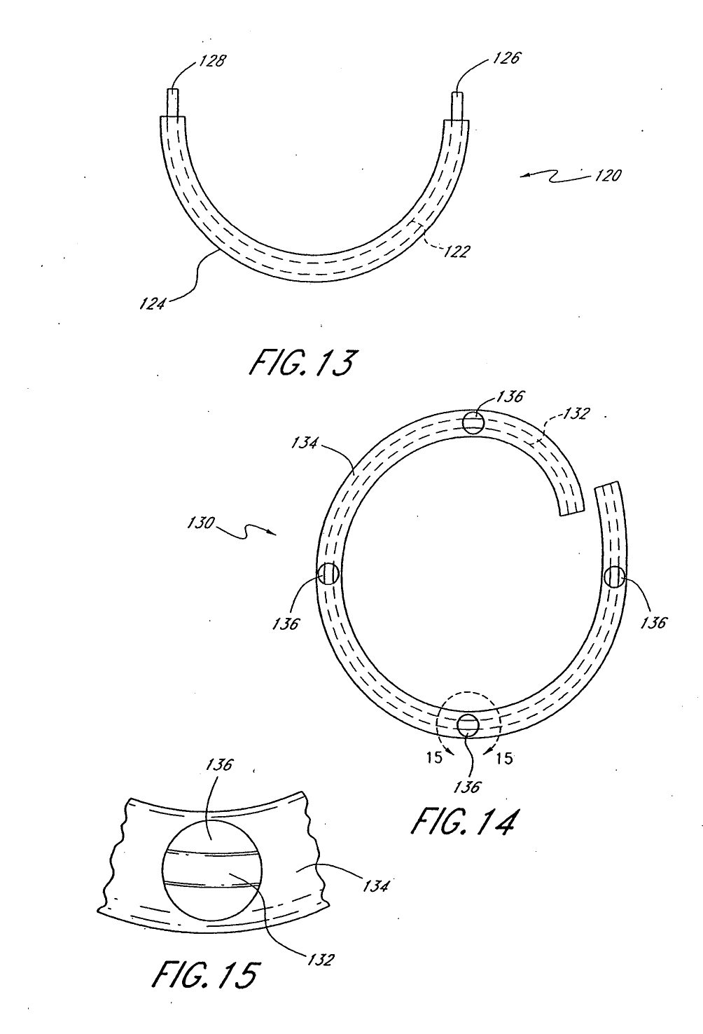

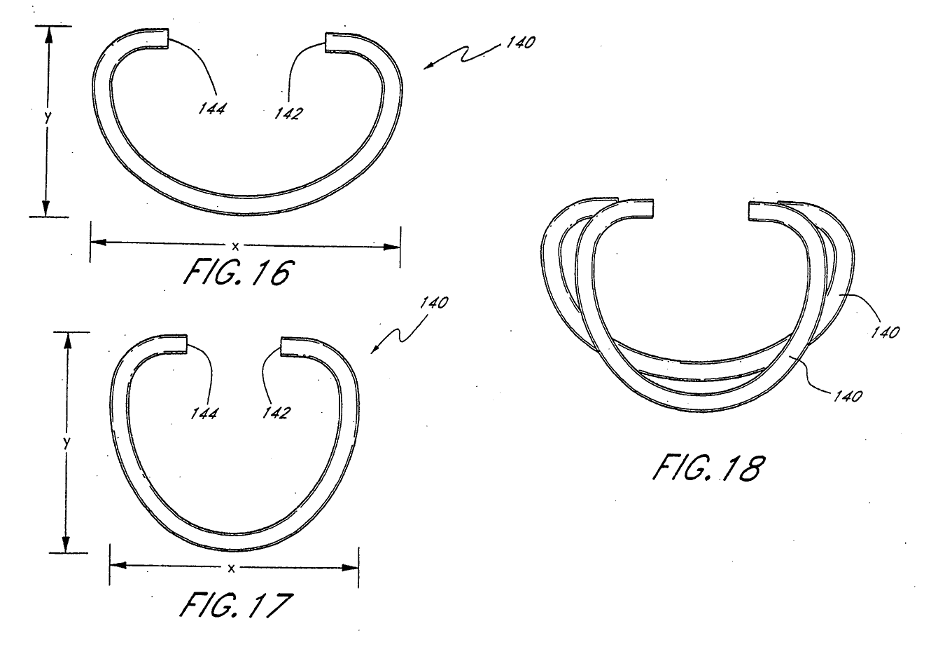

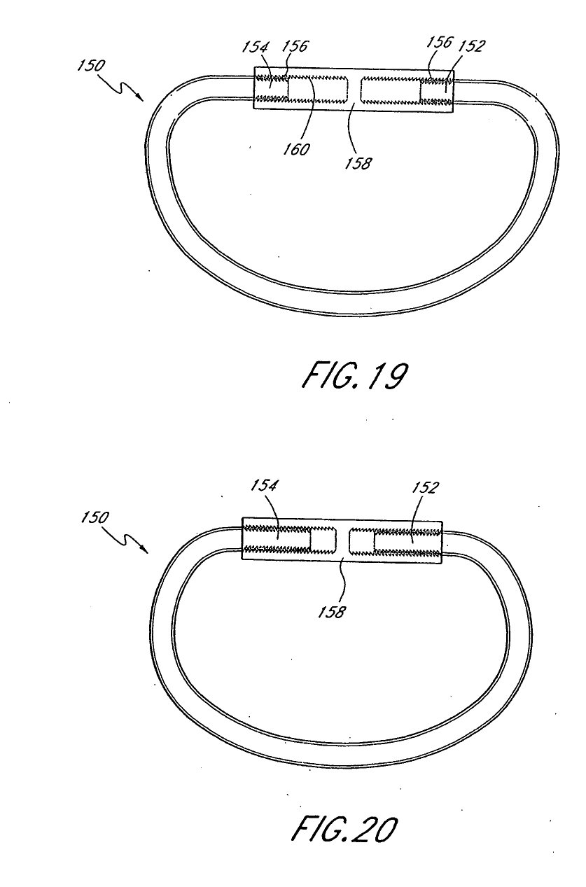

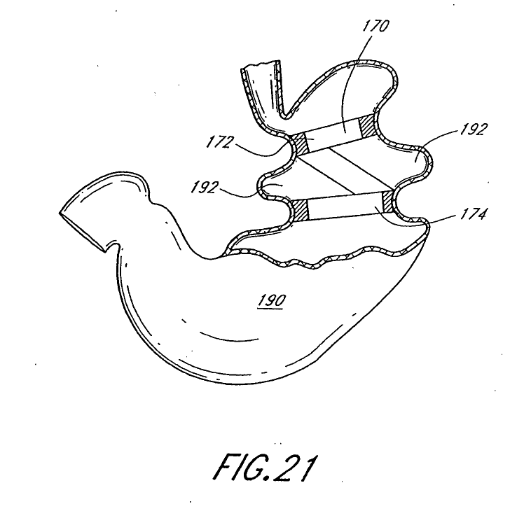

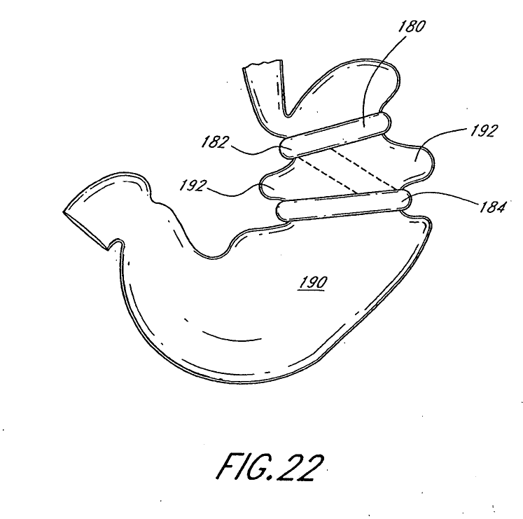

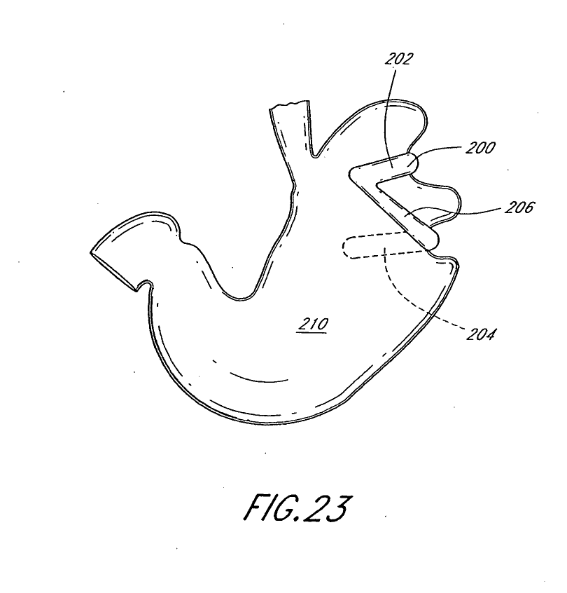

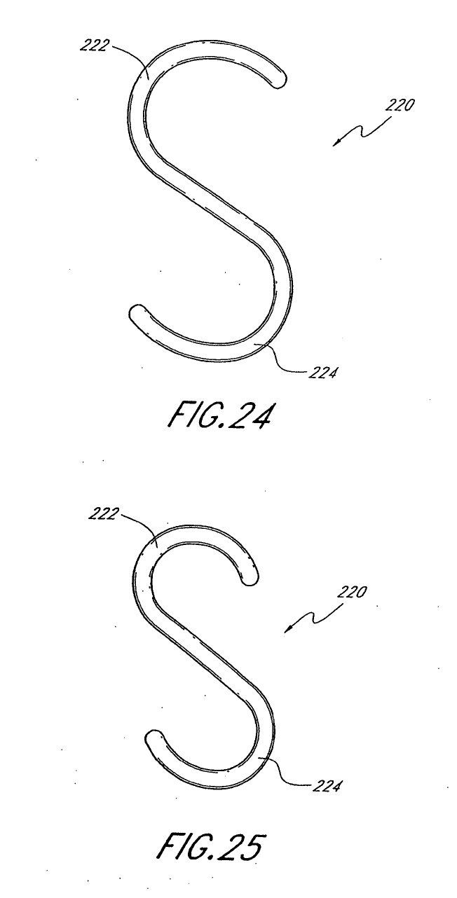

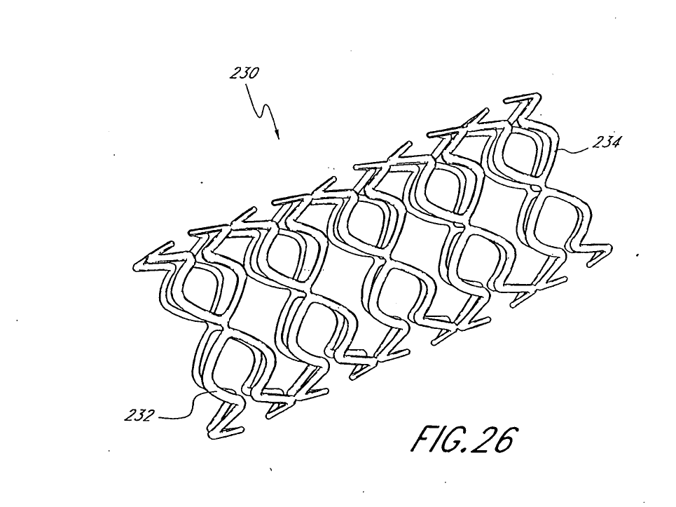

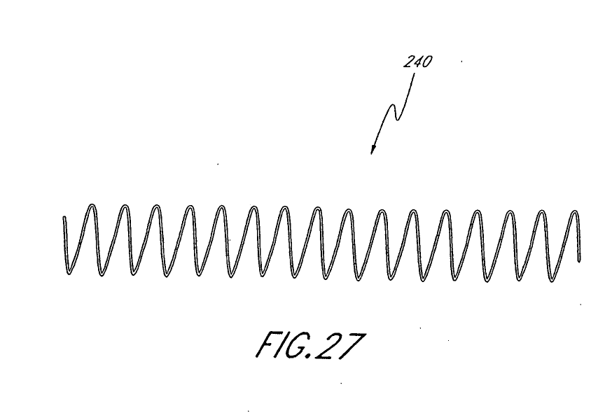

The present embodiments provide a system, method, and various devices to dynamically remodel and resize the stomach as the patient's needs change. For example, FIGS. 2 and 3 illustrate the pre- and post-adjustment configurations of a stomach 60 and one embodiment of a generally ring-shaped implant 62. In FIGS. 2 and 3 the implant 62 is configured to be disposed around the exterior surfaces of the stomach 60. FIG. 4 illustrates the pre-adjustment configuration of a stomach 60 and another embodiment of a generally ring-shaped implant 64 that is configured to be disposed within the stomach 60. The size and shape of each implant 62, 64 can be selected based upon the patient's anatomy. FIGS. 5-29, discussed in detail below, illustrate some examples of possible shapes.

FIGS. 2 and 4 illustrate the implants immediately after implantation, prior to any adjustments in the size and/or shape of the implants. In the illustrated configuration each of the generally ring-shaped implants forms a dividing line that separates the stomach into two regions. An upper region 66 includes the fundus, at least a portion of the cardia, and a portion of the body. A lower region 68 includes a portion of the body and the pylorus. Those of ordinary skill in the art will appreciate that the implants may be positioned and oriented in any of a variety of different ways from that illustrated. The exact positioning and orientation of the implants can be determined by the implanting physician according to the patient's needs.

The position of the implant relative to the stomach can be secured in any of a variety of ways. For example, sutures, staples, tacks, pins, and/or adhesives may secure the implant to the stomach. Stapling methods may include automatic or manual stapling. Adhesives may include, for example, tissue glue, heat activated glue, UV-curable glue, and room temperature or moisture activated glue. Securing and/or suturing of the various implant embodiments to the tissue can include a variety of energy sources, such as RF heating, laser, microwave, ultrasound, etc. Securing and/or suturing of the various implant embodiments to the tissue can be done all around the implant perimeter or at one or more points or segments. In certain embodiments, the implant may include one or more holes or suture rings through which sutures may pass, as described in more detail below.

FIG. 3 illustrates the stomach 60 and the external implant 62 of FIG. 2 after adjustments have been made to the size of the implant. As in FIG. 2, the generally ring-shaped implant separates the stomach into an upper region 66 and a lower region 68. The upper region forms a gastric pouch that can only hold a small amount of food. A stoma (not shown) connects the upper and lower regions. As the size of the implant decreases from the configuration of FIG. 2 to that of FIG. 3, the size of the stoma shrinks, thus limiting the rate at which food can pass from the upper stomach pouch to the lower region. Depending upon the patient's needs, the physician can activate the implant to achieve a smaller size, and thus a smaller stoma, from that illustrated in FIG. 3. Alternatively, during the activation procedure(s) the physician can stop short of the size illustrated in FIG. 3 so that the implant is configured to have a larger size, and thus a larger stoma, from that illustrated. As those of skill in the art will appreciate, the stomach and the internal implant 64 of FIG. 4 can be manipulated in a fashion similar to that just described for the external implant of FIGS. 2 and 3.

In certain embodiments the shape memory material of the implant may be bi-directional, so that it is capable of expanding and contracting. With such an embodiment, the physician can dynamically adjust the size and/or shape of the implant as the patient's needs change. For example, a patient may have a need to lose a large amount of weight quickly. In such a case it may be advantageous to shrink the implant down to a relatively small size soon after implantation. The relatively small implant would then create a relatively small stoma so that the speed at which the patient could digest food would be greatly diminished, and the patient would lose weight relatively quickly. As the patient loses weight, his or her needs may change, and the physician may need to expand the implant to create a larger stoma, and thereby increase the speed at which the patient can digest food. With a bi-directional implant, the physician could easily expand the implant using one or more of the non-invasive techniques described above.

FIGS. 5-7 illustrate one embodiment of a generally ring-shaped implant 70 that may be used in the methods described above and illustrated in FIGS. 2-4. The implant 70 comprises a ring with a male end 72 that telescopically engages a female end 74. FIGS. 5-7 represent a possible time-lapse transformation of the implant 70 from a deformed shape (FIG. 5) to a memorized shape (FIG. 7). As an activating energy (such as heat, or a magnetic field, or any of the other energies described above) is applied to the implant of FIG. 5, the circumference of the implant becomes progressively smaller as the implant returns to its memorized shape, shown in FIG. 7. As the implant becomes progressively smaller, it cinches the portion of the stomach around which it is wrapped, decreasing the size of the stoma that connects the upper gastric pouch to the lower stomach region. In order to achieve a desired circumference for the implant after it has been implanted, and thus achieve a desired circumference for the stoma, the physician may halt the application of activation energy before the implant returns to its memorized shape. For example, the application of activation energy may be halted when the implant occupies the intermediate configuration of FIG. 6.

In the illustrated embodiment, the implant 70 includes retaining features that help the implant to maintain its shape after the application of activation energy has ceased. The female end 74 includes a plurality of evenly spaced holes 76. The male end 72 includes at least one protrusion 78. As activation energy is applied to the implant 70, and it contracts from the configuration of FIG. 5 toward the configuration of FIG. 7, the at least one protrusion 78 advances from one hole 76 to the next along the female end 74 as the male end 72 advances into the female end. Engagement of the at least one protrusion with each hole resists any tendency of the male end to withdraw from the female end. These retaining features thus help the implant 70 to remain in its contracted state even as the contracted stomach and/or esophagus apply pressure against the implant that might otherwise cause the implant to expand toward the configuration of FIG. 5. If the implant includes a plurality of protrusions 78 and holes 76, as illustrated, then an increasing number of protrusions and holes will engage one another as the male end advances into the female end. As the number of engaged features increases, so does the retaining power of the implant.

Those of ordinary skill in the art will appreciate that the implant 70 shown in FIGS. 5-7 is representative of a family of implants having a generally ring-shaped configuration. A variety of implants having a generally ring-shaped configuration could be produced to meet the needs of a wide variety of patients. For example, a generally ring-shaped implant may include ends that do not telescope or even overlap. FIGS. 8 and 9 illustrate another embodiment of a generally ring-shaped implant 80. The implant 80 resembles the implant shown in FIGS. 5-7, and includes first and second ends 82, 84 that overlap, but are not in contact with one another. FIG. 8 illustrates a pre-activation configuration, while FIG. 9 illustrates a post-activation configuration. As the implant 80 transforms from the pre-activation configuration (FIG. 8) to the post-activation configuration (FIG. 9), an amount of overlap of the ends 82, 84 increases as a circumference of the implant tightens.

All of the embodiments of implants described herein may include features that facilitate the securement of the implant to the stomach and/or esophagus. For example, FIGS. 10-12 illustrate further embodiments of an implant 90, 100, 110 that is shaped substantially as an oval ring with overlapping ends. The implant 90 of FIG. 10 includes four evenly spaced suture holes 92, and the implant 100 of FIG. 11 includes four evenly spaced suture rings 102. In the illustrated embodiments, a longitudinal axis of each suture hole/ring extends in a direction substantially perpendicular to a plane defined by the implant. However, those of skill in the art will appreciate that the holes/rings could be oriented differently with respect to the implant. Each hole/ring may receive one or more sutures that may be used to secure the implant to the stomach. Those of ordinary skill in the art will appreciate that fewer or more suture holes/rings may be provided, and that they need not be evenly spaced. Those of ordinary skill in the art will also appreciate that suture holes/rings may be used with any of the implants described herein, and with implants of any shape or size.

The implant 110 of FIG. 12 includes four evenly spaced hooks or barbs 112. Each hook or barb includes a sharp point that is adapted to penetrate and grip tissue. The hooks or barbs thus secure the implant 110 to the stomach. Those of ordinary skill in the art will appreciate that fewer or more hooks or barbs may be provided, and that they need not be evenly spaced. Those of ordinary skill in the art will also appreciate that hooks or barbs may be used with any of the implants described herein, and with implants of any shape or size.

All of the embodiments of implants described herein may also include a cover. For example, FIG. 13 illustrates another embodiment of an implant 120 that is shaped substantially as a half ring, and FIG. 14 illustrates another embodiment of an implant 130 that is shaped substantially as a coiled ring with overlapping ends. Each implant 120, 130 includes a core 122, 132 formed of a shape memory material and a cover 124, 134 disposed over the core. The cover 124, 134 may be constructed of any biodegradable and/or biocompatible material, such as polytetrafluoroethylene (PTFE) and expanded polytetrafluoroethylene (ePTFE). The cover may include multiple layers, such as an insulating layer and a polymer jacket. The cover may serve as a protective barrier between the core and any surrounding tissue, and may help the implant to become integrated into the surrounding tissue. For example, the cover 124, 134 may be constructed of a porous material or a fabric. Such porous materials or fabrics can be impregnated with a time-release substance, such as anti-inflammatory drugs, anti-obesity drugs, a combination thereof, or other drugs. The cover may also comprise a lubricious coating, such as polylactic acid (PLA), that eases placement and/or removal of the implant. The cover may also aid in suturing the implant to the tissue by acting as a medium that sutures can penetrate. A surgeon implanting one of the present implant embodiments may pass a suturing needle first through the cover and then through the tissue to secure the implant to the tissue.

Depending upon the composition of the cover, it may insulate the core so that the core is less readily able to absorb activating energy and undergo a shape change. Accordingly, in the embodiment 120 of FIG. 13 at a first end and a second end of the implant the core 122 extends beyond the cover 124 to form a first exposed core portion 126 and a second exposed core portion 128. Similarly, in the embodiment of FIG. 14, the cover 134 includes four evenly spaced openings 136 that expose short lengths of the core 132. FIG. 15 illustrates a detail view of one of the openings 136 and the core 132. The exposed portions of the core may create locations where the core is readily able to absorb activating energy, which can then be conducted along the core to the non-exposed portions. The exposed portions thus provide locations at which activation energy can be focused, which both reduces energy loss during activation and reduces the likelihood that surrounding tissue might absorb unfocused activation energy and become damaged through overheating. In addition, any tissue in contact with an insulated portion of the implant is protected from absorbing heat through conduction from the implant.

FIGS. 16 and 17 illustrate another embodiment of a generally ring-shaped implant 140. The implant resembles the letter C, and includes first and second ends 142, 144 that do not overlap one another. FIG. 16 illustrates a pre-activation configuration for the implant 140, while FIG. 17 illustrates a post-activation configuration. In one embodiment of a method of implantation, the implant may be implanted in the pre-activation configuration, and then activated to induce a shape change. The activation may take the form of any of the methods described above, or any equivalent method.

In the pre-activation configuration, the implant includes a width dimension x and a height dimension y. As FIG. 18 illustrates, in the post-activation configuration the width dimension x of the implant is decreased, while the height dimension y of the implant is increased. Thus, no matter where the implant is placed on or in the stomach and/or esophagus, it reshapes and resizes the stomach and/or the esophagus to alter a path of travel of food through these areas, and/or to alter a patient's ability to absorb nutrients.

FIGS. 19 and 20 illustrate another embodiment of a generally ring-shaped implant 150. The implant 150 is similar in shape to the implant 140 shown in FIGS. 16 and 17, and includes first and second ends 152, 154 that do not overlap. FIG. 19 illustrates a pre-activation configuration, while FIG. 20 illustrates a post-activation configuration. Each of the implant ends 152, 154 includes ratchet teeth 156. A ratchet sleeve 158 receives each of the ends 152, 154. The sleeve 158 includes ratchet teeth 160 that are complementary to the teeth 156 on the implant ends. Thus, as the implant 150 progresses from the pre-activation configuration to the post-activation configuration the implant ends 152, 154 advance into the sleeve 158, and the mating ratchet teeth 156, 160 resist any tendency of the ends 152, 154 to withdraw from the sleeve 158. Because the implant ends are held firmly in the sleeve, there is less likelihood that the implant might relax and cause an unwanted change in shape of the stomach and/or esophagus.

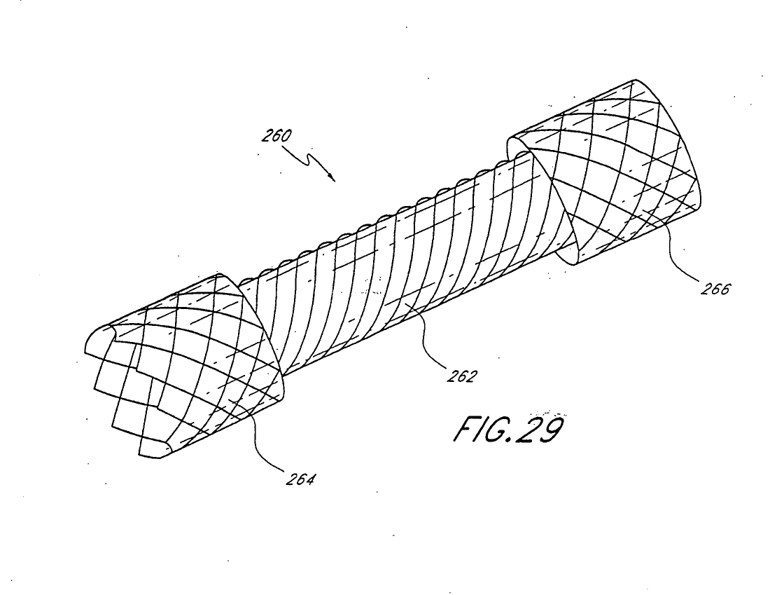

FIG. 30 illustrates, schematically, one possible configuration for implanting any of the implants of FIGS. 26-29. FIG. 30 shows a schematic configuration of an implant 270, the esophagus 272 and the stomach 274 shortly after implantation, and before any activation energy has been applied to the implant 270. In the illustrated embodiment, the implant 270 is located at the junction of the esophagus 272 and the stomach 274. An upper end 276 of the implant is located below the esophageal sphincter, while a lower end 278 of the implant extends into the stomach. Either end of the implant may be secured to the organ tissue, while portions of the implant in between the ends may also be secured to the tissue. While the illustrated implant is located within the esophagus and the stomach, those of skill in the art will appreciate that the implant could be located around the outside of these organs. Those of skill in the art will appreciate that any of the implants disclosed herein could also be located at the junction of the esophagus and the stomach. Those of skill in the art will also appreciate that the implants of FIGS. 26-29 could be implanted entirely within the stomach, or around the outside of the stomach.

When activation energy is applied to the implant 270 shown in FIG. 30, it may contract, thereby constricting the stomach/esophagus to narrow the food passageway and alter a path of travel of food through the stomach/esophagus. The extent of organ tissue constricted depends upon how much of the implant is secured to the stomach/esophagus.

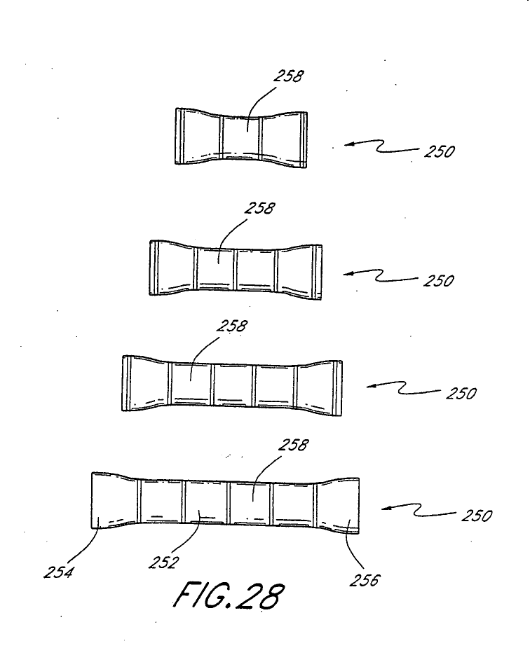

In FIG. 26, the implant 230 has a constant diameter from a first end 232 to a second end 234. In FIG. 28, the implant 250 has a constant diameter along an intermediate segment 252, then flares outwardly to a larger diameter at either end 254, 256. In FIG. 29, the implant 260 has a constant diameter along an intermediate segment 262, then abruptly transitions to a larger diameter at either end 264, 266. With the implants 250, 260 of FIGS. 28 and 29, the transition from the large opening at the proximal end 254, 264 to the relatively small intermediate section 252, 262 allows the implants to bring food slowly into the stomach, since the food will slow down at the bottleneck. Food will also exit the implant more quickly through the relatively wide distal end 256, 266.

Possible dimensions for the generally tubular implants of FIGS. 26-29 include the following. If the implant is to be positioned at the junction of the esophagus and the stomach, the implant might be between 5 mm and 50 mm in diameter, and between 20 and 200 mm in length. If the implant is to be positioned within or around the outside of the stomach, the implant might be between 20 mm and 100 mm in diameter, and between 20 and 200 mm in length.

In the embodiment 250 of FIG. 28, several different lengths of the implant are shown, and the cage-like structure of the implant is concealed by a sleeve 258. The sleeve 258 is analogous to the cover discussed above with respect to the embodiments having a shape memory core and a cover. The sleeve 258 may thus be constructed of any of the materials discussed above with respect to the cover, and share any of the same properties discussed above with respect to the cover.

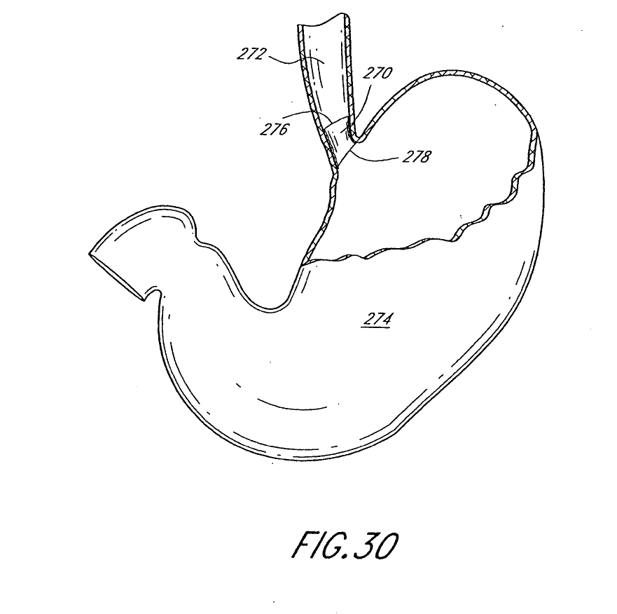

FIGS. 31 and 32 illustrate one possible configuration for any of the implants disclosed herein. The implant segment 280 includes a frame 282 constructed of a material that does not have a shape memory. For example, the frame 282 could be constructed of a metal or a polymer. Along an interior surface (a surface that will contact the stomach/esophagus) the frame 282 includes band 284 of a flexible material. For example, the band 284 could be constructed of silicone rubber. Disposed just behind the band is a layer of a shape memory material 286. In the illustrated embodiment, the shape memory material has a coiled configuration. However, those of skill in the art will appreciate that the shape memory material layer could have any configuration.

FIG. 31 illustrates the implant segment 280 in a pre-adjusted configuration, while FIG. 32 illustrates the implant segment 280 in a post-adjusted configuration. In FIG. 31 the inner band 284 is substantially flush with the inner surface of the frame 282. After the shape memory material 286 is activated, the inner band 284 is pushed outward away from the inner surface and into the configuration shown in FIG. 32. If an implant having the configuration of FIGS. 31 and 32 is disposed around the outside of a stomach/esophagus, the inner band 284 will constrict the stomach/esophagus as it is pushed away from the inner surface.

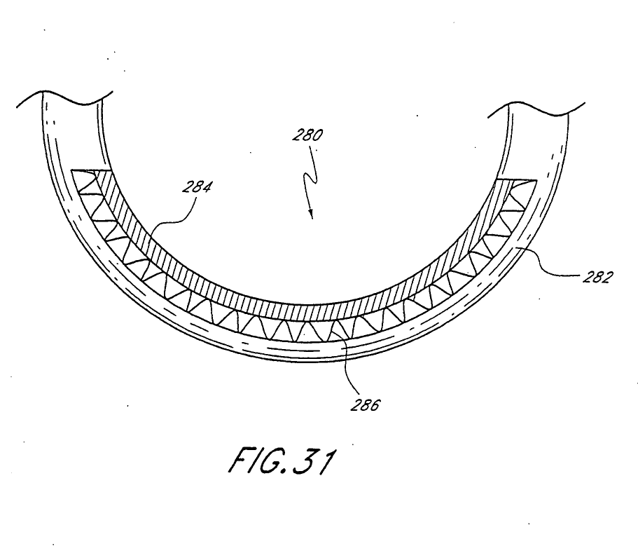

As discussed above, the size and/or configuration of any of the present implants may be adjusted post-implantation through one of many techniques, including minimally invasive techniques (endoscopic, laparoscopic, percutaneous, etc.) and completely non-invasive techniques (MRI, HIFU, inductive heating, a combination of these methods, etc.). FIG. 33 illustrates one example of a minimally invasive technique. The implant 290 may be directly connected to an electrical lead 292 that passes through the patient's skin. An external end of the lead may be connected to an electronic device 294 that is configured to generate electrical impulses. The lead 292 may transmit the impulses to the implant 290, generating activation energy within the implant in the form of heat.

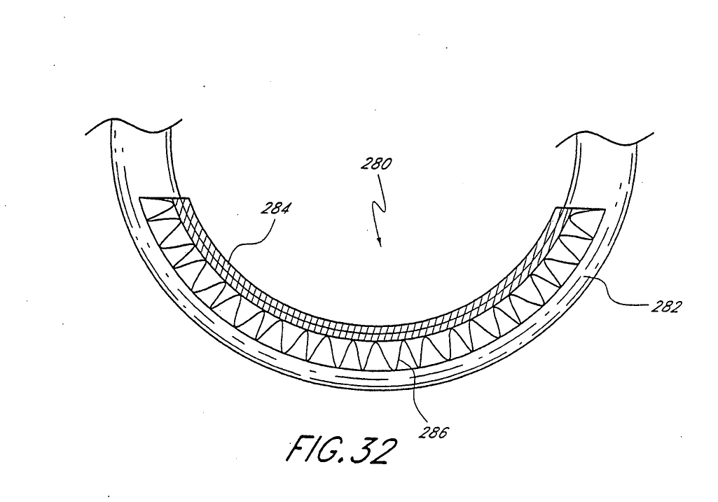

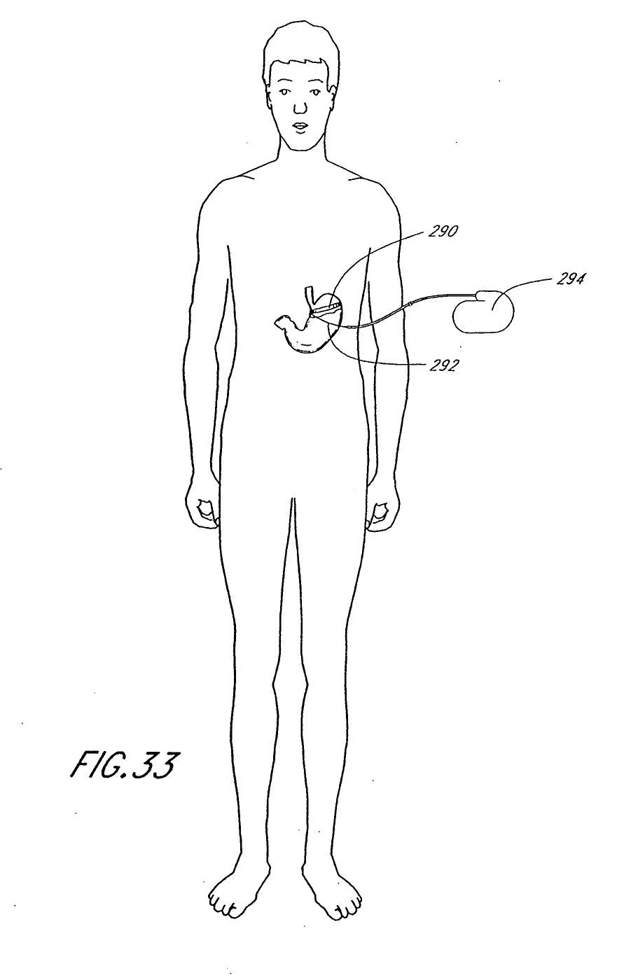

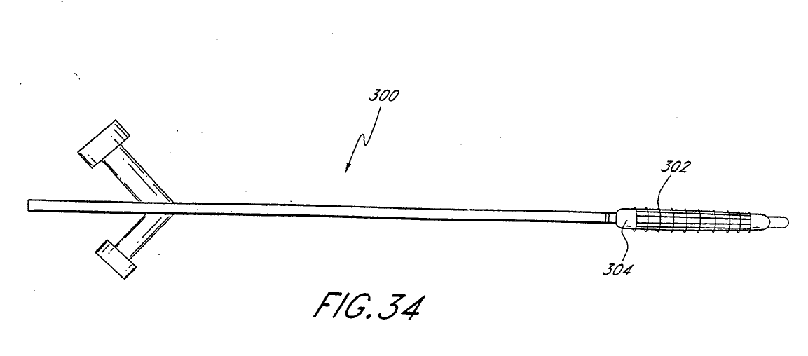

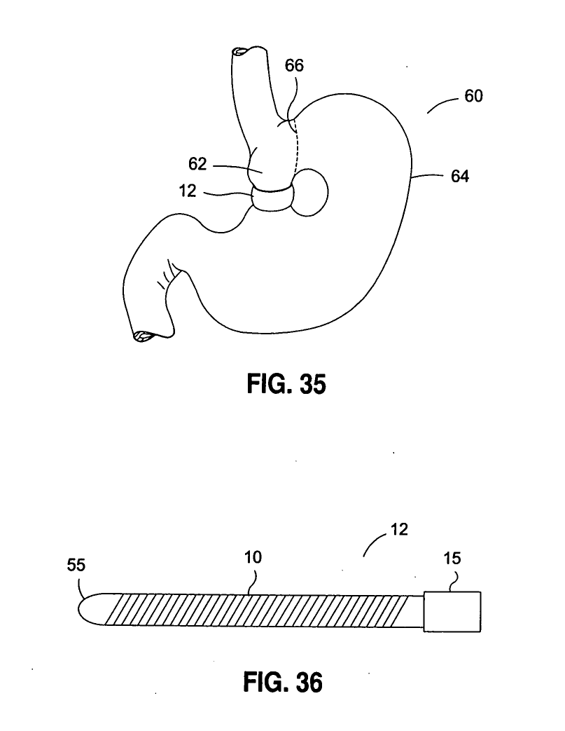

In certain embodiments, as shown in FIG. 35, an adjustable gastroplasty ring 12 may implanted into the body of a patient in conjunction with a vertical banded gastroplasty procedure. The adjustable implant may be disposed around a portion the stomach, or within the stomach to form an outlet from the pouch to the rest of the stomach. Here, a small pouch 62 may be made against the inner curve of the stomach 60 by vertically stapling 66 an upper portion of the stomach near the esophagus. The adjustable band 12 may then be positioned around the opening of the pouch 62 into the rest of the stomach 60. The implant may then be adjusted after implantation to control the size of the stoma, or opening, between the upper pouch 62 and the rest of the stomach 64.

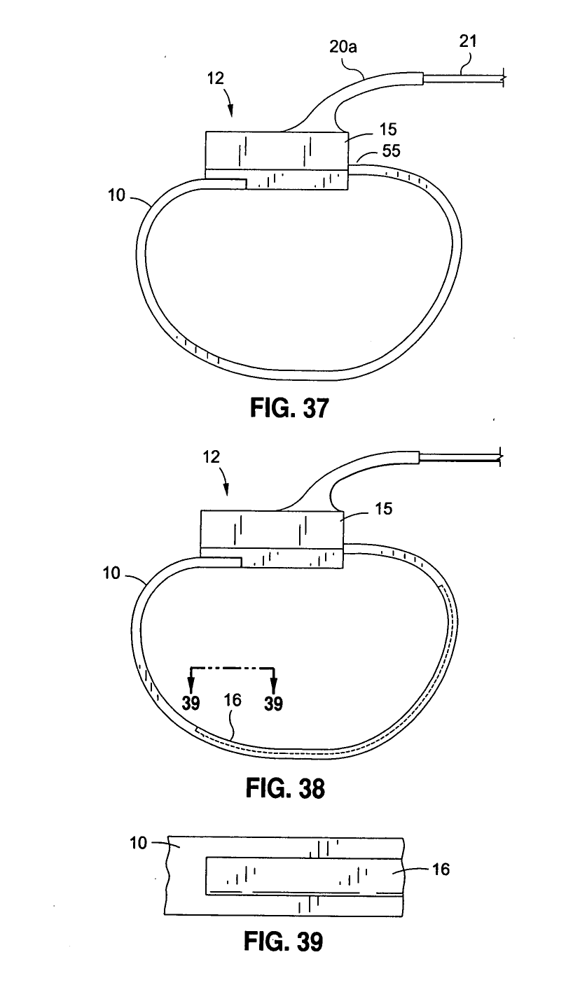

The implant may be implanted through an incision during a traditional open procedure, such as a laparatomy, or endoscopically, or laparoscopically, or percutaneously, or through another type of procedure, as those of skill in the art will appreciate. In certain embodiments, the implant may comprise a pre-implantation and a post implantation shape. In the pre-implantation shape, as shown in FIG. 36, the implant may comprise an elongate band 10 having a first end comprising a latch mechanism 15 and a second end 55 configured to be inserted into the latch mechanism 15 on the first end. In the pre-implantation shape, the implant may be laparoscopically or endoscopically positioned inside the patient's abdominal cavity near the patient's stomach. The surgeon may then manipulate the band into a loop surrounding the stomach, as shown in FIG. 37, by inserting the second end 55 of the band into the latch mechanism 15 on the first end of the band. The elongate band 10 may be manipulated to form a complete, closed loop wherein the first and second ends overlap or a discontinuous loop wherein the gap between the first and second ends is bridged and connected by the latching mechanism.

FIG. 35 illustrates the stomach 60 and the external implant 12 of FIG. 38 after the implant has been manipulated to form a loop surrounding the opening from the gastric pouch 62. The generally ring-shaped implant separates the stomach 60 into a gastric pouch 62 and a lower region 64. The gastric pouch 62 can only hold a small amount of food. A stoma (not shown) connects the gastric pouch 62 and lower region 64. As the size of the gastric band 12 is decreased, the size of the stoma shrinks, thus limiting the rate at which food can pass from the upper stomach pouch 62 to the lower region 64. Depending upon the patient's needs, the physician can activate the implant to achieve a smaller size, and thus a smaller stoma. Alternatively, during the activation procedure(s) the physician can stop short of the size illustrated in FIG. 35 so that the implant is configured to have a larger size, and thus a larger stoma, from that illustrated.

In certain embodiments the implant may be bi-directional, so that it is capable of expanding and contracting. With such an embodiment, the physician can dynamically adjust the size and/or shape of the implant as the patient's needs change. For example, a patient may have a need to lose a large amount of weight quickly. In such a case it may be advantageous to shrink the implant down to a relatively small size soon after implantation. The relatively small implant would then create a relatively small stoma so that the speed at which the patient could empty the gastric pouch and thus ingest food would be greatly diminished, and the patient would lose weight relatively quickly. As the patient loses weight, his or her needs may change, and the physician may need to expand the implant to create a larger stoma, and thereby increase the speed at which the patient can ingest food. With a bi-directional implant, the physician could easily expand the implant using one or more of the non-invasive techniques described below.

FIGS. 38-44 illustrate one embodiment of a bidirectional gastroplasty band 12 that may be used in the methods described above and illustrated in FIG. 35. As depicted in FIG. 38, the adjustable gastroplasty band 12 may comprise a band 10, made from a nylon plastic, or any other suitable plastic polymer, having a latch head 15 mounted on one end. The latch head 15 houses the working mechanism of the gastroplasty band 12. For example, in certain embodiments, the working mechanism may comprise an actuator for moving the nylon band 10 through the latch head 15. Here, the nylon band 10 comprises a plurality of detents 11 along one surface. The actuator 29 is configured to constrict the gastroplasty band 12 by successively engaging the detents 11 on the nylon band 10 to feed the band through the latch head and thereby reduce the diameter of the gastroplasty band 12. As shown in FIGS. 38-39, a spring release 16 may be mounted on the band 10 and biased to return the gastroplasty band 12 to its fully released position when the actuator is released. The installed shape or loop of the band can be seen in FIG. 38, this would be considered the “as” implanted shape and/or fully released position.

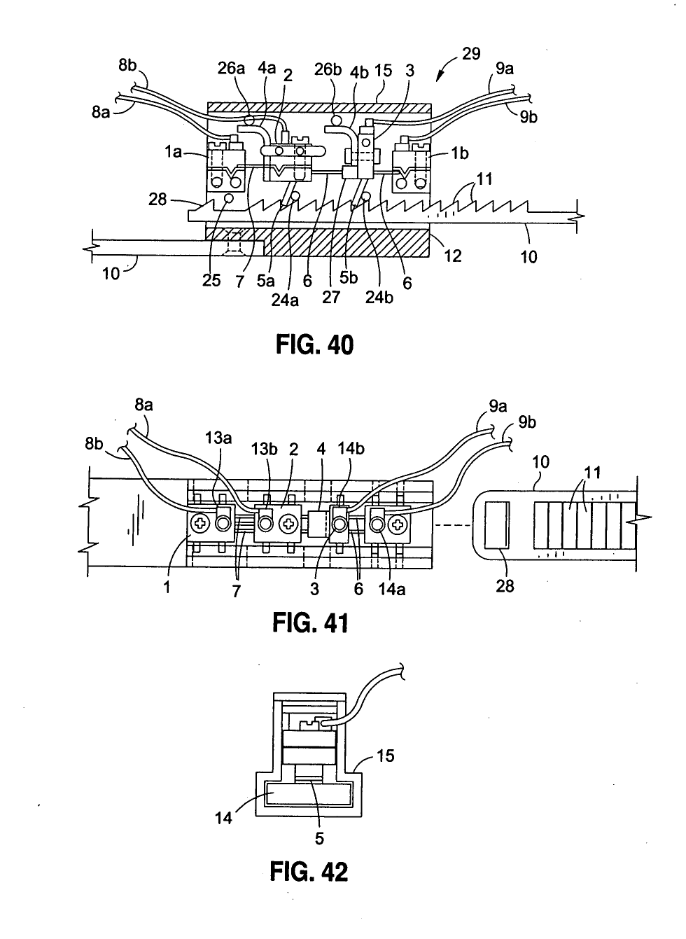

As shown in FIGS. 40-42, the actuator comprises an indexing shuttle 2 and a holding pawl assembly 3 connected by a shape memory wire 6. The indexing shuttle 2 has a second shape memory wire 7 extending from the opposite end of the indexing shuttle and connected to an anchor clamp 1a. The second shape memory wire may be comprised of the same shape memory alloy as the first shape memory wire. Alternatively, the second shape memory wire may be comprised of a different shape memory alloy than the first shape memory wire. The indexing shuttle 2 and holding pawl assembly 3 each have a nylon pawl 5a and 5b extending from the bottom of each assembly. The plastic pawls are a molded in features of the indexing shuttle 2 and the holding pawl assembly 3 and because of the elastic nature of the plastic pawls 5a and 5b, they are in constant contact with the nylon plastic band 10 and the detents 11 on the nylon band 10.

In use, when the second shape memory wire 7 is actuated, for example by heating, it enters an austenite phase and assumes a shape which pushes the indexing shuttle 2 towards the holding assembly 3 and the indexing pawl 5a is pushed up and over the adjacent detent 11, thereby incrementally taking up the nylon band 10 and reducing the diameter of the gastric band 12. The holding pawl 5b is likewise pushed up and over an adjacent detent 11. The holding pawl 5b engages the adjacent detent 11 and provides extra support for holding the band 10 in the desired diameter against the pressure of the forces from a return leaf spring 16 embedded in the gastroplasty band 12.

Once the pawls 5a and 5b have engaged the next detent 11, stainless steel return springs 4a and 4b, initially pushed against spring stop pins 26a and 26b as the indexing shuttle is pushed forward, return the indexing shuttle 2 and the holding pawl assembly 3 to their original positions. Preferably, the shape memory wire 7 has a diameter such that may quickly transform between its austenite and martensite phases and associated shapes. The shape memory wire then may be reactivated, for example by heating, to re-enter the austenite phase and push the indexing shuttle forward, thereby incrementally advancing the indexing pawl 5a over another detent 11 until the desired diameter for the gastroplasty band 12 is achieved.

The actuator further comprises a second shape memory wire 6 secured to nylon plastic anchor clamp 1b, passed through the holding pawl assembly 3 and then terminated into the indexing shuttle assembly 2. A locking collar 27 is clamped onto the shape memory wire 6 next to the holding pawl assembly 3. When the shape memory wire 6 is actuated it constricts, thereby pulling the indexing shuttle 2 and the holding pawl assembly 3 toward the two blocking pins 24a and 24b. The pawls 5a and 5b are pushed against the blocking pins 24a and 24b. The pawls 5a and 5b pivot against the blocking pins 24a and 24b and are pulled up and off of the band 10 and detents 11 on the band. With the pawls 5a and 5b no longer opposing the force of the leaf spring 16, the band 10 will retract from the latch assembly 15 until the band 10 stop pin 25 on the latch head 15 engages the stop detent 28 located on the end of the band 10. Once the pawls 5a and 5b are disconnected from the detents 11, the release spring 16, mounted on the band 10 (shown in FIGS. 38-39) will cause the band 10 to return to a fully open position. The stop detent 28 and the stop pin 25 prevent the band 10 from fully exiting the latch head 15.

As shown in FIGS. 40-41 and 43-44, actuation of the shape memory wires 6 and 7 is controlled by the power delivered through an inductive coil assembly 17. The inductive coil assembly 17 is connected to the latch head 15 of the gastroplasty band 12 via a wire harness 21. When the gastroplasty band 12 is implanted, the inductive coil assembly 17 is positioned underneath the patient's skin at the side of the stomach. In use, a second, matching inductive coil 18 may be placed over the location of the implanted inductive coil assembly 17 to transfer power via inductive coupling of the two coils. The signal power may then be sent down wire harness 21 and split off to the individual wires 8a and 8b, which are secured to anchor clamp 1a and indexing shuttle 2 at points 13a and 13b, or wires 9a and 9b which are secured to anchor clamp 1b and holding pawl assembly 3 at points 14a and 14b respectively. Power may be alternately supplied to wires 8a and 8b to activate shape memory wire 7, and to wires 9a and 9b to activate shape memory wire 6.

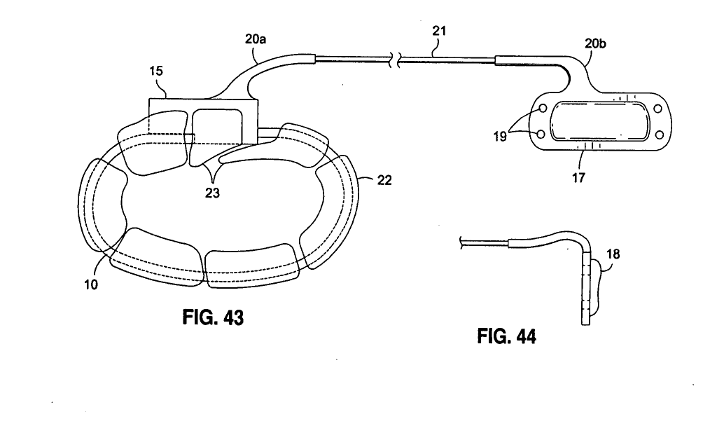

As shown in FIG. 43, the inductive coil assembly 17 is jacketed inside a tough silicone rubber skin. Four holes 19 on either side of the assembly provide locations for suture lines to pass through and anchor the assembly down. In certain embodiments, silicone rubber jackets may cover wire harness 21 and strain reliefs 20a and 20b to insulate surrounding tissue from the power transmitted along the wire harness 21.

In certain embodiments, as shown in FIG. 43, one or more silicone rubber pads 22 may be added to the band 10 to give the band a wider footing and soft edges that will keep the band 10 from cutting into the underlying tissue. The silicone pads 22 may also supply pressure points that will help with constriction of the stomach wall.

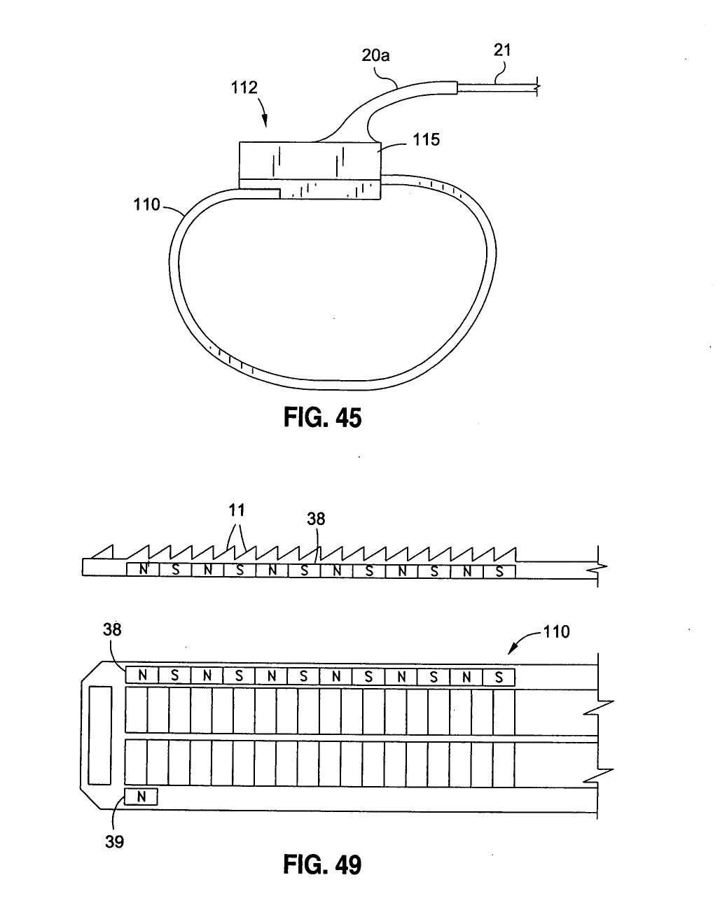

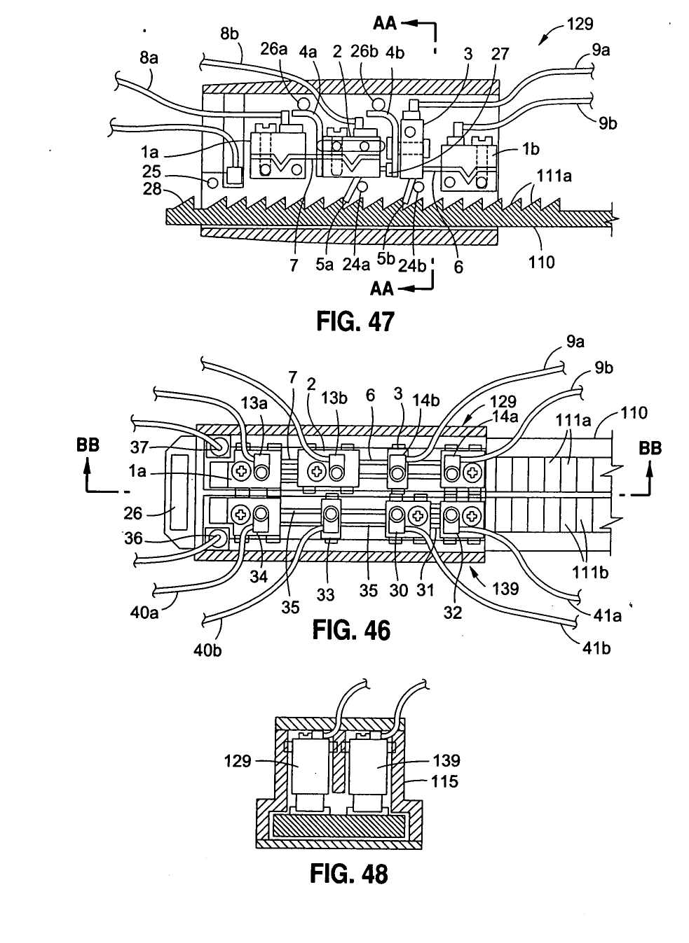

In an alternative embodiment, as shown in FIGS. 45-51, the gastroplasty band 112 may comprise may comprise a band 110, made from a nylon plastic, or any other suitable plastic polymer, having a latch head 115 mounted on one end. The latch head 115 comprises two actuators 129 and 139 for moving the nylon band 110 back and forth through the latch head 115. As shown in FIG. 46, the nylon band 110 comprises a plurality of detents 111a and 111b extending along one surface. The first set of detents 111a are angled in a first direction while the second set of detents 111b are angled in the opposite direction. A first actuator 129 is configured to constrict the gastroplasty band 112 by successively engaging the detents 111a on the nylon band 110 to feed the band through the latch head 115 and thereby reduce the diameter of the gastroplasty band 112. A second actuator 139, identical to the first actuator 129, but disposed in the opposite direction is configured to expand the gastroplasty band 112 by successively engaging the detents 111b on the nylon band to withdraw the band 110 from the latch head 115 and thereby expand the diameter of the gastroplasty band 112.

As shown in FIG. 47, the first actuator 129 is similar to the actuator 29 of the above described embodiment (shown in FIGS. 40-41). The actuator 129 comprises an indexing shuttle 2 and a holding pawl assembly 3 connected by a shape memory wire 6. The indexing shuttle 2 has a second shape memory wire 7 extending from the opposite end of the indexing shuttle and connected to an anchor clamp 1a. The indexing shuttle 2 and holding pawl shuttle each have a nylon pawl 5a and 5b extending from the bottom of each assembly. The plastic pawls 5a and 5b are molded in features of the indexing shuttle 2 and the hold-down assembly 3 and because of the elastic nature of the plastic pawls 5a and 5b, they are in constant contact with the nylon plastic band 110 and the detents 111a on the nylon band 110.

In use, the shape memory wire 7 is actuated and pushes the indexing shuttle 2 towards the holding assembly 3. The indexing pawl 5a is then pushed up and over the adjacent detent 111a, thereby incrementally taking up the nylon band 110 and reducing the diameter of the gastric band 112. The holding pawl 5b is likewise pushed up and over an adjacent detent 111a and engages the adjacent detent 111a to provides extra support for holding the band 110 in the desired diameter. Once the pawls 5a and 5b have engaged the next detents 111a, stainless steel return springs 4a and 4b, initially pushed against spring stop pins 26a and 26b as the indexing shuttle 2 is pushed forward, return the indexing shuttle 2 and the holding pawl assembly 3 to their original positions. The shape memory wire 7 then may be reactivated, for example by heating, to re-enter the austenite phase and push the indexing shuttle forward, thereby incrementally advancing the indexing pawl 5a over another detent 111a until the desired diameter for the gastroplasty band 112 is achieved.

As shown in FIG. 46, a second actuator 139 which may be a complete copy of the first actuator 129 only reversed, is mounted along side the first actuator 129 for indexing the band out of the latch head 115. When the shape memory wire 31 is activated, indexing shuttle 30 and holding assembly 33 are pushed forward and the pawls extending from the indexing shuttle 30 and holding assembly 33 are likewise pushed forward to engage successive detents 111b on the band 110 and incrementally withdraw the band 110 from the latch head 115.

However, in order for either of the actuators 129, 139 to be able to incrementally move the band 110 along their respective detents 111a, 111b on the band 110, the pawls of the non-working actuator must be disengaged from their detents 111a or 111b.

As shown in FIG. 47, with respect to the first actuator 129, each actuator further comprises a second shape memory wire 6 secured to a nylon plastic anchor clamp 1b and passed through the holding pawl assembly 3 and then terminated into the indexing shuttle assembly 2. A locking collar 27 is clamped onto one of the shape memory wires 6 next to the holding pawl assembly 3. When the shape memory wire 6 is actuated it pulls the indexing shuttle 2 and the holding pawl assembly 3 toward the two blocking pins 24a & 24b, this pushes the two pawls 5a and 5b up and off of the band 10 and detents 111a on the band 110, thus enabling the second actuator 139 to operate and withdraw the band 110 from the latch head 115 without resistance from the pawls 5a and 5b. Likewise, as shown in FIG. 46, a second shape memory wire 35 attached to anchor 34 pass in through holding assembly 33 and terminating at indexing shuttle 30 may be actuated to disengage the corresponding pawls on indexing shuttle 30 and holding assembly 33 when the first actuator is engaged to feed the band 110 into the latch head 115. As described above, detent 28 the stop pin 25 provide a safety feature for the band 110 by preventing the band 110 from being able fully exit the latch head 115.

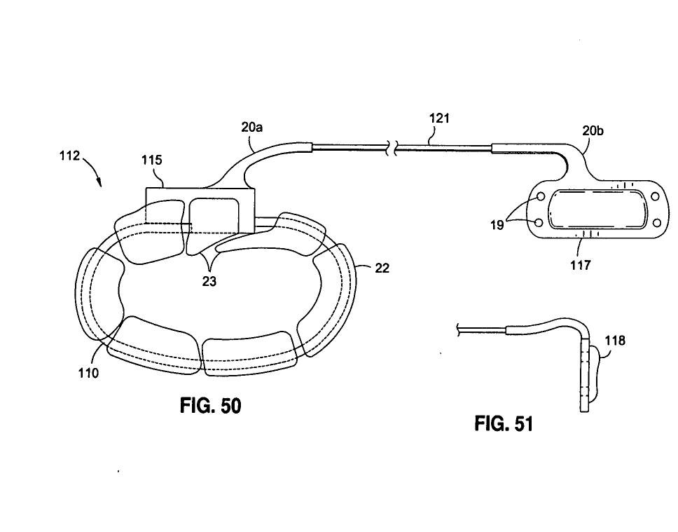

As shown in FIGS. 50 and 51, actuation of the shape memory wires 6, 7, 31 and 35 is controlled by the power delivered through an inductive coil assembly 117. The inductive coil assembly 117 is connected to the latch head 115 of the gastroplasty band 112 via a wire harness 121. When the gastroplasty band 112 is implanted, the inductive coil assembly 117 is positioned underneath the patient's skin at the side of the stomach. In use, a second, matching inductive coil 118 may be placed over the location of the implanted inductive coil assembly 117 to transfer power via inductive coupling of the two coils. The signal power may then be sent down wire harness 121 and split off to the individual wires 8a and 8b, which are secured to anchor clamp 1a and indexing shuttle 2 at points 13a and 13b, wires 9a and 9b which are secured to anchor clamp 1b and holding pawl assembly 3 at points 14a and 14b respectively, wires 40a and 40b which are secured to anchor clamp 34 and holding pawl assembly 33 or wires 41a and 41b which are secured to anchor clamp 32 and indexing shuttle 30. Power may be alternately supplied to wires 8a and 8b to activate shape memory wire 7 and wires 40a and 40b to disengage actuator 139 or to wires 41a and 41b to activate shape memory wire 31 and wires 9a and 9b to disengage actuator 129.

In certain embodiments, as shown in FIGS. 46 and 49, the gastroplasty band 112 may further comprise a position sensing element 37 located in the latch head 115 and a magnetic encoder strip 38 mounted on the band 110. The position sensing element 37 and the magnetic encoder strip 38 may form a position feedback loop that can be used to indicate the size of the loop opening. A second sensor 36 and magnetic trigger 39 are used to indicate the home positions, i.e. a fully released loop. This information may be sent through the wire harness 21 and inductive coil assembly 17. The information is then received and displayed to the doctor on a handheld instrument.

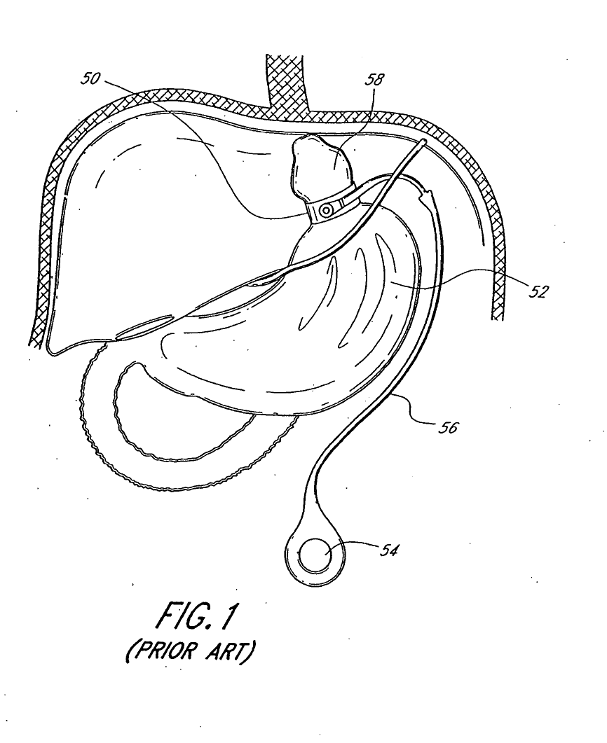

Also as discussed above, the present implants may be implanted in any of a variety of ways, such as during a traditional open procedure, or endoscopically, or laparoscopically, or percutaneously, or through another type of procedure. FIG. 34 illustrates one method of implanting the present implants using a balloon catheter 300. The implant 302 may be loaded over the balloon 304, and the balloon advanced to the implantation site. Once the implant reaches the implantation site, the balloon may be inflated to expand the implant. After the balloon is deflated and removed from the implantation site, the expanded implant can be secured to the stomach/esophagus using any of the methods described above. While FIG. 34 illustrates a generally tubular implant, those of skill in the art will appreciate that the balloon catheter implantation method can be used with any of the implants described herein. Further, embodiments such as those illustrated in FIGS. 35 through 51 can be used for a gastric band of the type depicted in FIG. 1.

The above presents a description of the best mode contemplated for carrying out the present gastric implants and methods, and of the manner and process of making and using them, in such full, clear, concise, and exact terms as to enable any person skilled in the art to which it pertains to make and use these gastric implants and methods. These gastric implants and methods are, however, susceptible to modifications and alternate constructions from that discussed above that are fully equivalent. Consequently, these gastric implants and methods are not limited to the particular embodiments disclosed. On the contrary, these gastric implants and methods cover all modifications and alternate constructions coming within the spirit and scope of the gastric implants and methods as generally expressed by the following claims, which particularly point out and distinctly claim the subject matter of the gastric implants and methods.

What is claimed is:

1. An adjustable gastric implant for constraining at least a portion of a stomach, comprising:
an elongate member having first and second ends, the elongate member configured to engage the stomach;
at least one actuator coupled to the first and second ends of the elongate member, wherein the at least one actuator comprises a shape memory material;
wherein activation of at least a portion of the shape memory material results in a conformational change in the at least one actuator; and
wherein the conformational change in the at least one actuator moves the elongate member from a first conformation to a second conformation, such that the first and second ends move with respect to each other, resulting in a change in a lumenal dimension of the stomach.

2. The implant of claim 1, wherein placement of the elongate member engages the stomach between an upper region and lower region connected by a stomal lumen.

3. The implant of claim 2, wherein moving the elongate member from a first conformation to a second conformation reduces a size of the stomal lumen.

4. The implant of claim 2, wherein moving the elongate member from a first conformation to a second conformation increases a size of the stomal lumen.

5. The implant of claim 1, wherein the implant is configured to be placed within the stomach.

6. The implant of claim 1, wherein the implant is configured to be placed around an outer surface of the stomach.

7. The implant of claim 1, wherein the activation comprises application of an energy to the shape memory material.

8. The implant of claim 7, wherein the energy is at least one of ultrasound energy, radio frequency energy, X-ray energy, microwave energy, light, electric field energy, magnetic field energy, inductive heating, or conductive heating.

9. A method of regulating food intake in a patient, comprising the steps of:
providing an adjustable gastric implant comprising an elongate member coupled to an actuator having a shape memory component;
placing the implant to engage at least a portion of the stomach between an upper region and a lower region connected by a stomal opening;
applying an activation energy to the shape memory component;
wherein application of the activation energy transforms the shape memory component from a first conformation to a second conformation, said transformation effective to drive the actuator; and
wherein driving the actuator results in a conformational change in the implant such that a diameter of the stomal opening is decreased; and
wherein decreasing the diameter of the stomal opening reduces the rate at which food passes through the stomach.

10. The method of claim 9, further comprising reconfiguring the shape memory component from the second conformation back to the first conformation.

11. The method of claim 10, further comprising alternating the conformation of the shape memory component between the first and second configurations to decrease incrementally a diameter of the stomal opening.

12. The method of claim 9, wherein the actuator engages the ends of the elongate member to form a substantially closed loop.

13. The method of claim 12, wherein the implant further comprises a bias member, and the method further comprises disengaging at least one end of the elongate member from the actuator, such that the bias member is effective to increase the perimeter of the closed loop formed by the elongate member to a maximal perimeter.

14. The method of claim 13, wherein the disengaging further comprises activating a second shape memory component on the actuator, thereby disengaging the actuator.

15. The method of claim 12, wherein the implant further comprises a second actuator having a third shape memory component, the second actuator coupled to the elongate member, the method further comprising:
applying an activation energy to the third shape memory component;
wherein application of the activation energy results in the third shape memory component being transformed from a first conformation to a second conformation; and
wherein transformation of the third shape memory component drives the second actuator to expand a perimeter of the loop resulting in an increase in the diameter of the stomal opening, thereby increasing a rate at which food can pass through the stomach.

16. The method of claim 15, wherein the shape memory component of the implant comprises at least one of a metal, a metal alloy, a nickel titanium alloy, and a shape memory polymer.

17. The method of claim 16, wherein a shape memory component of the implant comprises at least one of Fe—C, Fe—Pd, Fe—Mn—Si, Co—Mn, Fe—Co—Ni—Ti, Ni—Mn—Ga, Ni₂MnGa, and Co—Ni—Al.

18. The method of claim 9, wherein the activation energy comprises at least one of magnetic resonance imaging energy, high-intensity focused ultrasound energy, radio frequency energy, x-ray energy, microwave energy, light energy, electric field energy, magnetic field energy, inductive heating, and conductive heating.

19. A method of adjusting a gastric implant in a patient, comprising;
placing an adjustable gastric implant around at least a portion of the stomach of the patient;
adjusting the implant to produce a constriction of the stomach;
using at least one of a magnetic resonance imaging and ultrasound imaging technique to determine a first size of the constriction; and
adjusting the gastric restriction device to vary the constriction to a second size and limit the rate at which food passes through the constriction.

20. The method of claim 19, wherein the imaging technique comprises an ultrasound technique that uses speed of sound shift.
